(19) United States
(12) Patent Application Publication
(10) Pub. No.: US 2023/0028621 A1
(43) Pub. Date: Jan. 26, 2023

(54) Method, automated system and cartridge for extraction of cell-free nucleic acids from a blood sample

(71) Applicant: BiopSense Oy, Jyväskylä (FI)

(72) Inventors: Marja Tiirola, Jyväskylä (FI); Jussi Pennanen, Jyväskylä (FI); Veli-Mikko Puupponen, Jyväskylä (FI)

(21) Appl. No.: 17/785,438

(22) PCT Filed: Dec. 23, 2020
(86) PCT No.: PCT/FI2020/050866

§ 371 Date: Jun. 15, 2022

(30) Foreign Application Priority Data
Dec. 23, 2019 (FI) 20196132

(51) Int. Cl. C12N 15/10

(52) U.S. Cl. CPC C12N 15/1013; C12N 15/1017

(57) ABSTRACT

The present invention is directed to a method for extraction of cell-free nucleic acid fragments from a blood sample to facilitate cancer diagnosis, prognosis and monitoring as well as prenatal screening. The present invention provides a cartridge comprising a first compartment for filtering plasma from a blood sample and preferably also for cell fixation and cell rinsing in order to improve yield and a second compartment for performing nucleic acid separation, wherein the first compartment comprises a hollow fiber membrane and the second compartment comprises material for binding the nucleic acids or a gel for electrophoresis. The invention also provides and an automated system comprising a device with a docking site adapted to receive said cartridge, said device comprising means adapted to operate the blood plasma filtering process in said cartridge and means adapted to operate nucleic acid separation in said cartridge.

### FIELD OF THE INVENTION

The present invention is related to the field of liquid biopsy diagnostics. In particular, it relates to cancer diagnosis, prognosis and monitoring facilitated by the detection of circulating tumor-derived small nucleic acids in the bloodstream. The method is also useful in sample preparation for prenatal testing of maternal blood (i.e. Non Invasive Prenatal Test, NIPT).

### BACKGROUND OF THE INVENTION

Plasma and serum purified from blood includes variable amounts of molecules originating from self and non-self (placenta, tumors, transplants) tissues. For example, during pregnancy, the mother's blood contains both maternal DNA and fetal DNA, which originates from the placenta. Tumors, on the other hand, continually shed circulating tumor DNA (ctDNA), circulating tumor cells (CTC), exosomes, platelets, microRNAs, as well as protein markers into the bloodstream. The process for detecting these molecular signatures in blood samples or other body fluids is called liquid biopsy.

Blood cell-free DNA (cfDNA) is a valuable and less-invasive tool for healthcare, for studying the status (sex and aneuploidy) of fetus, aiding in diagnosis and monitoring of cancer, indicating cardiovascular and autoimmune diseases, and showing physical exercise, transplants and even sepsis (1). Genetic and epigenetic changes of the cfDNA as well as fragmentation and simply the quantity are valuable information collected from the cfDNA analysis (1).

However, the non-self fraction of cell-free nucleic acids in the bloodstream is low, and cfDNA/cell-free RNA (cfRNA) does not remain stable for long periods in regular blood samples, but can be contaminated with self DNA or be degraded. In prenatal screening, fetal fraction derived from apoptosis of villous cytotrophoplasts forms less than 4% of the maternal plasma cell-free DNA at 11-13 weeks' gestation (2). In cancer patients, ctDNA is released through a lysis of apoptotic and necrotic cells, digestion of tumor cells by macrophages, or by direct secretion of DNA by live tumor cells (3), and varying between 0.01 and 90% of the total cfDNA fraction (4). This level depends on tumor stage, vascularization, burden, biological features, such as apoptotic rate, and metastatic potential of the cancer cells, and the factors affecting the patient's blood volume affects the concentration (5). Changes in the ctDNA mutation frequencies (% VAF, variant allele frequency) have been observed as soon as two days after lung cancer operations (6). In addition, not only the mutation frequency, but also the total amount of ctDNA can be used as a tool on a treatment response evaluation, or as a follow-up marker on early detection of relapse following primary surgery. For example, post-operative ctDNA findings predicted relapse in 93% of patients, with median lead time of 60 days prior to radiological confirmation (7). Also, methylation status of cancer cells differs from normal cells, which can affect the properties of cancer-derived cfDNA (8). The half-life of ctDNA in the blood stream varies between 16 min to 2.5 h, making ctDNA a “real time” biomarker reflecting the tumor burden (4,9). Liquid biopsies have notable advantages over tissue biopsies as they provide information on the complete heterogeneity (both spatial and temporal), sampling procedures are minimally invasive or totally non-invasive, and repeated sampling is possible for following up treatment efficacy, development of resistance, and cancer progression. Moreover, it is predicted that liquid biopsy can form a less expensive and fast sample preparation (10).

The first pre-analytical step for the preparation of the cfDNA from a blood sample is separation of the red and white blood cells, which may hamper the analysis. Although serum is a common material for clinical studies, cfDNA is usually extracted from plasma, due to lower non-tumor background of wild type DNA, released from the white blood cells (11). It is well known that plasma separation is critical step in performing the liquid biopsy analysis (12). If any white blood cells are present in the plasma sample, they will release wild type DNA and cause dilution of the cfDNA sample. In current procedures for cfDNA extraction, separation of the plasma is done using centrifugation of the stabilized blood. Improper shipping and storage conditions for the blood sample may damage the cells and release DNA and DNA nucleases, resulting in genomic wild type DNA contamination and loss or reduction in ctDNA content (13). One commercialized solution for shipping blood samples for later plasma isolation and processing is the use of specific cell-free DNA blood collection tubes (provided e.g. by Streck Inc., Roche and PreAnalytiX), which may retain sample stability for up to 7-14 days when stored at room temperature (RT; 15-25° C.). However, climate-controlled transportation is expensive and especially important in boreal and arctic areas, where freezing can completely destroy sensitive samples. In warmer areas, cooling packs may be used, but expedited shipping is often necessary and temperature fluctuations still pose a risk. Furthermore, centrifugation of the blood samples for plasma separation is time consuming, whether it happens at the site of sample collection or in the analytical laboratory. Special care should be practiced to avoid the disruption of blood cells, since it would yield to leakage of cellular nucleic acids to the sample. Centrifugation of the blood often needs to be performed twice at two different centrifugation forces, depending on the sample stabilizing reagent (such as EDTA or aldehydes) and extraction protocol. Every liquid transfer between the tubes increases the risk of human errors, sample cross-contamination and potential biohazard to the personnel, if the patient has a blood-borne disease. After plasma separation, the sample can be stored frozen for downstream biomarker isolation.

American Society of Clinical Oncology (ASCO) panel reviewed (14) that the processing of K₂EDTA stabilization liquid biopsy tubes should be as expedient as possible, within 6 hours from collection, to avoid lysis of white blood cells, which can dilute the ctDNA with normal leukocyte DNA and release DNA nucleases. The use of leukocyte stabilization tubes can allow greater flexibility in the time to processing of up to 48 hours, or longer with some tubes, without compromise of ctDNA detection or quantification. Furthermore, ASCO panel critically stated that collection, handling variables, storage condition, and patient-related biological factors were all considered factors that hamper liquid biopsy diagnostics.

The size distribution of circulating cfDNA fragments have often been contradictory and malignant conditions have been reported to result in both increased (15) and decreased (16) overall cfDNA size. Indeed, Mouliere et al. (16) showed that up to 98% of the ctDNA in colorectal cancer patient was made up by <400 bp fragments. The size distribution of non-tumor derived cfDNA was more even, and <400 bp fragments constitute approximately 65% of these nucleic acids. Short circulating tumor-derived nucleic acid molecules (DNA/RNA) of less than 400 bp can thus be useful in cancer diagnosis and for monitoring drug response in cancer therapy.

Some of the observed low congruence in CLIA-certified commercially available liquid biopsy tests (17) may be due to sample handling problems that might be avoided using automation. Automation is currently possible in laboratory processes in which samples can wait in the queue, or if the number of retrieved samples enables continuous high-throughput processing. Separation of plasma from the red and white blood cells is done by centrifugation, which can be automated in high-volume central laboratories with sampling robots, but in smaller laboratories, it requires precise manual work and trained personnel. Nucleic acids (and proteins) can be further isolated by, for example, purification with magnetic particles or solid-phase filter columns. This step can be performed using automated or semi-automated sample handling equipment, which are fairly expensive and require batch processing for economical usage. Semi-automated combination of plasma separation/nucleic acid extraction is available in batch mode in some expensive high-throughput systems (e.g. Vanadis Extract by Perkin Elmer), but still requires external blood centrifugation.

Commercialized, fully automated solution, especially for use in the “low-sample-number” patient-side settings do not yet exist, but some implementations have been described in the literature. However, they still rely on manual sample loading and analyte collection, which necessitates trained laboratory personnel and leads to unnecessary risk of sample contamination, loss, mix-up and transmission of blood-borne diseases. In addition, these methods are able to only process limited sample volumes (<5 ml blood, <2 ml plasma) (e.g. 18), which is a severe limitation considering the detection of rare mutations. Optimally, liquid biopsy sample processing should be fully automated, reproducible, support large sample volumes and capable for asynchronous processing start times, immediately after a blood draw has been taken.

Nucleic acid and protein extraction devices frequently use functionalized columns or magnetic beads due to their customizable surface chemistry and simple, fluidic or non-fluidic magnetic processing. Nucleic acid purification methods using magnetic beads are thus well-known in the art. Automated devices are typically based on a plate or chip, and they use an external magnet to either keep the beads stationary within a plate or move either the beads or the fluid through an array. These devices require either complex fluid transfer systems or movable robotics, and they are currently optimal only for high-volume batch-based processing.

In the prior art of U.S. Pat. Nos. 6,802,820 and 6,802,971, there are disclosed specialized hollow fiber membranes having unique and highly efficient asymmetric fiber wall characteristics capable of separating plasma from whole blood in-vivo by exposing the exterior surface of the fiber to whole blood and directing plasma through the fiber wall. After the plasma is treated for removal of waste products, excess fluids, toxins, and/or other deleterious plasma proteins, the treated plasma is returned and reintroduced to the patients' blood stream.

WO2019/185874 relates to the use of hollow fibers, in particular polyethersulfone hollow fibers having a porosity above 20 nm, to impoverish blood and blood-derivatives like plasma from blood-derived extracellular vesicles, in particular microvesicles and exosomes and exomers, and to methods for obtaining and analysing such impoverished samples using gravity filtering.

WO2019/136086 discloses a simple tangential flow filtration unit with hollow fiber membrane for the separation of cells and parts thereof, bacteria, viruses and the like from a biological fluid. The unit includes a collection chamber in fluid communication with the filtration unit, wherein a vacuum draws the biological fluid through the apparatus, and wherein selected biological material comprised by the biological fluid is separated from a remainder of the biological fluid.

US20040050699 discloses a cassette for use in performing electrophoretic separations of nucleic acids on a solid substrate. The cassette includes reservoirs for running buffer, electrodes for receiving electrical current and a substrate support. The cassette is designed to connect with external fluid and electrical sources. A device for use in performing electrophoresis separation is also disclosed. The device is of modular construction with a docking station for receiving the cassette.

U.S. Pat. No. 5,863,801A discloses a cassette for automated extraction of nucleic acids from several biological samples in parallel operation using paramagnetic particles with affinity for nucleic acids. The cassette includes hollow needles for sample loading, valves, reservoirs for beads, wash solutions and nucleic acid extraction fluid and a separable sample transfer strip. The cassette is designed to be operated in a related device providing docking slots for the cassettes.

In EP2315849, an approach to quantify circulating tumor DNA (ctDNA) in plasma and fecal samples of patients is disclosed. In the method, blood samples are subjected to centrifugation in order to separate plasma. It is disclosed that measurements of ctDNA can be used to reliably monitor tumor dynamics in subjects with cancer, especially those who are undergoing surgery or chemotherapy.

However, there is still a growing need in the art for novel methods facilitating a personalized approach for assessing and treating disease in subjects with cancer.

### SUMMARY OF THE INVENTION

The solution provided by the present invention is based on a disposable cartridge, where a combination of means for pressure-aided plasma filtration using hollow fiber membrane and means for subsequent biomarker extraction enables fully automated blood biomarker isolation. In the present invention, a cell fixation reagent contained by the cartridge and added to the sample prior to cell separation effectively minimizes the leakage of cellular genomic DNA to plasma. Further, the washing step(s) performed for the separated cell fraction maximizes the yield of cfDNA. Isolated cell-free biomarkers (cfDNA, cfRNA and cell-free proteins) can be further analyzed from the purified sample or the sample can be safely sent to a diagnostic service provider over regular mail. A binder-based extraction of nucleic acids, such as use of magnetic beads, is a suitable method in the biomarker purification and concentration. Gel electrophoresis and filter extraction are alternative embodiments. The method and cartridge are fully automatic avoiding the need of trained laboratory staff and enabling safe processing of individual blood samples in “low-sample number” settings. As a result, the method enables immediate sample processing and provides an encapsulated, stable concentrated nucleic acid sample that tolerates all transport conditions, including freezing and high temperatures. In addition, the repeatability of the method is superior to manual sampling, and constant high yields are achieved.

Accordingly, in an aspect, the present invention provides a method for extraction of nucleic acid fragments from a blood sample, the method comprising the steps of:

a) providing a blood sample taken from an individual;

b) preferably stabilizing blood cells in the sample with a fixative reagent;

c) filtering the blood sample in order to separate plasma from said blood sample by using a hollow fiber filter;

d) preferably washing the fraction comprising blood cells obtained from step c) with a wash solution and collecting the washout;

e) contacting the plasma sample obtained in step b), and preferably the washout collected from step d), with a binder material specific to nucleic acids, or alternatively subjecting the plasma sample, and preferably the washout collected in step d), to electrophoresis in a separating medium, in order to purify nucleic acid fragments present in said plasma sample, wherein said separating medium is prepared so that nucleic acid fragments are capable to migrate in the medium and can be separated in said medium by size;

f) collecting those nucleic acid fragments bound to said binder material or which have migrated in said separating medium; and

g) mixing the nucleic acid fragments collected in step f) with a preservative or a stabilizing agent;

wherein the steps b) to g) are performed on an automated system on a cartridge comprising a first compartment for filtering plasma from a blood sample and a second compartment for contacting the plasma sample obtained in step c) with a binder material specific to nucleic acids, or alternatively said second compartment comprises means for performing an electrophoresis. In a preferred embodiment, the cartridge comprises means to add reagents such as enzymes to plasma, washed cells or the washout collected in step d) in order to separate or purify cfDNA from vesicles, proteins or other biomolecules or debris originating from the blood sample.

In certain aspects, the present invention provides a cartridge comprising a first compartment for filtering plasma from a blood sample and a second compartment for contacting a plasma sample with a binder material specific to nucleic acids, wherein the first compartment comprises a hollow fiber filter and the second compartment comprises a chamber for nucleic acid purification, and said cartridge comprises, encapsulated in said cartridge, a binder material specific to nucleic acids, wherein said binder material preferably comprises magnetic beads.

In certain aspects, the present invention provides an automated system for extraction of nucleic acid fragments from a blood sample comprising a device with a docking site adapted to receive the cartridge as defined above, said device comprising means adapted to operate the blood plasma filtering process in said cartridge and means adapted to operate nucleic acid purification in said cartridge with a binder material specific to nucleic acids.

In certain aspects, the present invention provides a cartridge comprising a first compartment for filtering plasma from a blood sample and a second compartment for performing gel electrophoresis, wherein the first compartment comprises a hollow fiber membrane and the second compartment comprises a gel for electrophoresis.

In certain aspects, the present invention provides an automated system for extraction of nucleic acid fragments from a blood sample comprising a device with a docking site adapted to receive the cartridge as defined above comprising a gel for electrophoresis, said device comprising means adapted to operate the blood plasma filtering process in said cartridge and means adapted to operate gel electrophoresis in said cartridge.

In certain aspects, the present invention provides a method for extraction of protein biomarkers from a blood sample, the method comprising the steps of:

a) providing a blood sample taken from an individual;

b) preferably stabilizing blood cells in the sample with a fixative reagent;

c) filtering the blood sample in order to separate plasma from said blood sample by using a hollow fiber filter;

d) preferably washing the separated cell fraction with a washing reagent and collecting the washout;

e) contacting the plasma sample obtained in step c), and preferably the washout collected in step d), with a binder material specific to a protein biomarker in order to purify said biomarker whenever present in said plasma sample;

f) collecting those proteins bound to said binder material; and

g) mixing the proteins collected in step f) with a preservative or a stabilizing agent;

wherein the steps b) to g) are performed on an automated system on a cartridge comprising a first compartment for filtering plasma from a blood sample and a second compartment for contacting the plasma sample obtained in step c) with a binder material specific to said protein biomarker.

In certain aspects, the present invention provides a cartridge comprising a first compartment for filtering plasma from a blood sample and a second compartment for contacting a plasma sample with a binder material specific to a protein biomarker, wherein the first compartment comprises a hollow fiber filter and the second compartment comprises a chamber for protein purification, and said cartridge comprises a binder material specific to said protein biomarker encapsulated in said cartridge, wherein said binder material preferably comprises magnetic beads.

In certain aspects, the present invention provides an automated system for extraction of protein biomarker from a blood sample comprising a device with a docking site adapted to receive the cartridge as defined above comprising binder material specific to a protein biomarker, said device comprising means adapted to operate the blood plasma filtering process in said cartridge and means adapted to operate protein purification in said cartridge with a binder material specific to said protein biomarker.

### DESCRIPTION OF THE DRAWINGS

FIG. 1. Preferred process steps for using and operating the cartridge of the invention for extraction of circulating small nucleic acids from a blood sample.

FIG. 2. The exploded view of a preferred cartridge structure combining plasma extraction with gel electrophoresis.

FIG. 3. The exploded view of a preferred cartridge structure combining plasma extraction with magnetic bead extraction.

FIG. 4. Flow diagram showing fluidics of the cartridge structure using gel electrophoresis.

FIG. 5. Flow diagram showing fluidics of the cartridge structure using magnetic bead extraction in combination with peristaltic pump system.

FIG. 6. Flow diagram showing fluidics of the cartridge structure using magnetic bead extraction in combination with gas pressure pumping for plasma separation.

FIG. 7. Flow diagram showing fluidics of the cartridge structure using magnetic bead extraction in combination with gas pressure pumping system.

FIG. 8. Schematic drawing showing the assembly of the gel electrophoresis equipment in the cartridge structure.

FIG. 9. Schematic drawing showing alternative presentations a-d of passive mixing configuration detail 24 in FIG. 5-7

FIG. 10. Schematic drawing showing alternative top view presentations a-f of fluidic magnetic bead trapping configuration detail 87 in FIG. 5-7

FIG. 11. Schematic drawing showing alternative side view presentations g-k of fluidic magnetic bead trapping configuration detail 87 in FIG. 5-7

FIG. 12. Exploded view showing alternative structure for mechanical blade stirrer container detail 20 in FIG. 5-7

FIG. 13. Exploded view showing alternative structure for magnetic stirrer container detail 20 in FIG. 5-7

FIG. 14. Schematic drawing showing top view presentation of removable sample storage container detail 87 in FIG. 2-3

FIG. 15. Schematic drawing showing fluorescence reader configuration for measuring sample nucleic acid concentration.

FIG. 16. Shows electropherograms of representative DNA samples prepared from plasma extracted using filtration and two-step centrifugation as described in Example 2.

FIG. 17. Shows electropherogram of a DNA sample prepared from plasma extracted using single-step centrifugation as described in Example 2.

FIG. 18. Shows a fluorescence microphotograph of the magnetic bead fluorescence measurement as described in Example 5.

FIG. 19. Shows a graph of measured fluorescence intensity as a function of DNA concentration on magnetic beads as described in Example 5.

FIG. 20. Yield for four randomly selected samples is shown. The average cfDNA yield, when employing a cell washing step, was found to be 155% relative to the samples processed without this step, demonstrating improved sample recovery.

FIG. 21. The structural view of another preferred cartridge design combining plasma extraction with magnetic bead extraction.

FIG. 22. Detailed structural view of the sample tube interface and channel arrangement of the design in FIG. 21.

### DETAILED EMBODIMENTS

The problem solved by the present invention is provision of an efficient and simple method for isolation and storage of cell-free nucleic acids or protein biomarkers from bloodstream. The present invention is the first to disclose that by combining plasma separation, and preferably cell fixation, and cell washing/elution, with nucleic acid extraction through electrophoretic separation or non-mechanical fluidic manipulation of a solid-phase binder material, it is possible to design a process that can be performed in a small-scale automated cartridge for the extraction of circulating nucleic acids/protein biomarkers from a blood sample. The present method is intended to alleviate shortcomings in the prior art by providing convenient sample collection, automatic quantification, transport, storage as well as high yields from large volume samples and good stability for the isolated nucleic acids.

In particular, the present invention provides a method comprising the steps of:

a) providing a blood sample taken from an individual;

b) preferably stabilizing blood cells in the sample with a fixative reagent;

c) filtering the blood sample in order to separate plasma from said blood sample by using a hollow fiber filter;

d) preferably washing the fraction comprising blood cells obtained from step c) with a wash solution and collecting the washout;

e) contacting the plasma sample obtained in step b), and preferably the washout collected from step d), with a binder material specific to nucleic acids, or alternatively subjecting the plasma sample, and preferably the washout collected in step d), to electrophoresis in a separating medium, in order to purify nucleic acid fragments present in said plasma sample, wherein said separating medium is prepared so that nucleic acid fragments are capable to migrate in the medium and can be separated in said medium by size;

f) collecting those nucleic acid fragments bound to said binder material or which have migrated in said separating medium; and

g) mixing the nucleic acid fragments collected in step f) with a preservative or a stabilizing agent;

wherein the steps b) to g) are performed on an automated system on a cartridge comprising a first compartment for filtering plasma from a blood sample and a second compartment for contacting the plasma sample obtained in step c) with a binder material specific to nucleic acids, or alternatively said second compartment comprises means for performing an electrophoresis.

In a preferred embodiment, the cartridge comprises means to add reagents such as enzymes to plasma, washed cells or the washout collected in step d) in order to separate or purify cfDNA from vesicles, proteins or other biomolecules or debris originating from the blood sample. The cell-free DNA in a plasma preparation is mostly associated with histone proteins and contained within extracellular vesicles. To facilitate the effective recovery of the DNA, a pre-treatment with a proteolytic enzyme, e.g. proteinase K, or other highly promiscuous proteinase in suitable buffer (lysis buffer) conditions is necessary to release the nucleic acids. In case of the application for recovery and purification of intact protein markers from the plasma, the application of mild denaturing conditions in the form of suitable surfactants and other chaotropic agents is appropriate.

Preferably, the length of said nucleic acid fragments collected in step d) of the above method is less than 400 bp, more preferably 100-200 bp. This is in line of the biological processes disclosed to be involved in circulating tumor DNA (ctDNA) release including apoptosis and necrosis from dying cells. The size of fragmented cell-free DNA has been shown to be mainly about 166 bp long.

In a preferred embodiment, the blood sample obtained in step a) of the above method is mixed with an anticoagulant before subjecting the sample to the filtering step b). Preferred anticoagulants can be selected from the group consisting of: heparin and derivatives thereof, ethylenediaminetetraacetic acid (EDTA) and coumarin derivatives such as warfarin.

In a preferred embodiment, cell fixation reagent is added to the blood sample in step b) in order to enhance the separation of cells from the plasma and to prevent the leakage of cellular nucleic acids to plasma. “Cell fixation” refers herein to a technique that maintains the structure of cells and/or sub-cellular components such as cell organelles (e.g., nucleus). Fixing modifies the chemical or biological structure cellular components by, e.g., cross-linking them. Fixing may cause whole cells and cellular organelles to resist lysis. Of special interest herein, fixing may also cause cellular nucleic acids to resist release into a surrounding medium. For example, fixing may prevent nuclear DNA from white blood cells to resist release into a plasma fraction during centrifugation of whole blood. “Cell fixation reagent” or “fixative” refers herein to an agent such as a chemical or biological reagent that fixes cellular nucleic acids and thereby causes cells to resist release of such nucleic acids into a surrounding medium. A cell fixation reagent may disable cellular proteolytic enzymes and nucleases. Examples of fixatives include aldehydes (e.g., formaldehyde), alcohols, and oxidizing agents. Preferred cell fixation reagents include cross-linking fixatives are selected from the group consisting of: aldehydes and their derivatives (e.g. formaldehyde, glutaraldehyde), azines (e.g. 1,3,5-Triacryloyl-hexahydro-s-triazine) or hemiacetals (e.g. 2,3-dimethoxytetrahydrofuran).

Preferably, said hollow fiber membrane utilized in step c) is composed of a single material phase which material is a chain high polymer with low nucleic acid or protein binding capacity, such as cellulose acetate, polypropylene or PVDF and is capable of forming fibers by the spinning of the high polymer. The production of hollow fiber membranes are disclosed, e.g., in U.S. Pat. No. 4,234,431. In another aspect, the hollow fibers may include a mixed cellulose ester, polysulfone, plastic polymers or a combination thereof. In another aspect, the hollow fibers may include at least one of a hydrophobic material, a hydrophilic material, a low-nucleic-acid-binding material or a low-protein-binding material. They may include pore sizes ranging from about 0.5 microns to about 5 microns, depending on the intended use or uses. They may have various densities ranging from 50 to 500.

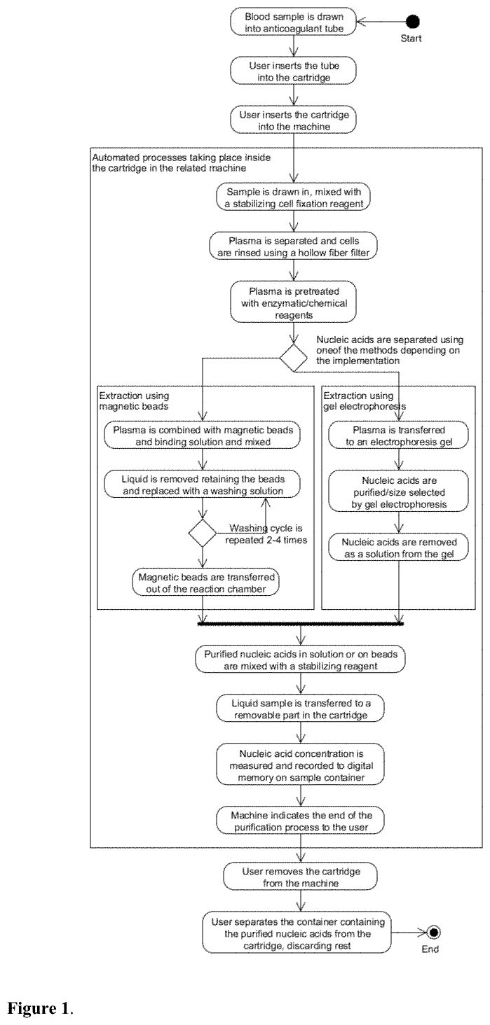

In another preferred embodiment, said separating medium in step e) of the above method is a size-selecting medium for nucleic acid fragments. The concentration of the medium determines the pore size of the medium which affects the migration of DNA. Increasing the concentration of the medium reduces the migration speed and improves separation of smaller DNA molecules. The optional electrophoresis in step e) of the above method is preferably performed in a container filled with a running buffer, an electrophoresis gel matrix portion that is configured within the container, and a loading well configured within said separating medium. The separating medium may extend laterally across the container. The separating medium can be agarose, or it may be at least one of starch, agar, agarose, and polyacrylamide. The running buffer may have a pH between pH 7 and pH 9, and it may comprise EDTA as a chelating agent. The container may also comprise at least one positive electrode and at least one negative electrode. The rate of migration of the DNA is proportional to the voltage applied, i.e. the higher the voltage, the faster the DNA moves. Apparatuses and systems for electrophoretic nucleic acid sample preparation are disclosed, e.g., in WO2016061416. Process steps of the preferred embodiments of the above-described method are outlined in FIG. 1.

The present invention is further directed to cartridge comprising means adapted to perform the above method in combination with an external device providing power, pressure (such as compressed air or gas) and/or vacuum for the cartridge. The cartridge preferably comprises two compartments or layers, the first comprising fluidics and liquid reagents for plasma separation, and preferably also for cell fixation and cell rinsing/washing. The second layer contains a chamber for nucleic acid purification or an electrophoresis gel, running buffer solution and related electrodes. The cartridge further contains means for retention of the isolated nucleic acid sample. The casing covers the cartridge from the top, bottom and sides and comprises openings for valves, power supply, pumps and blood sample tube. The scale of the cartridge or the casing is such that the volume is preferably between 150-1500 cm³ or 150-1000 cm³, more preferably 150-500 cm³, and most preferably 150-250 cm³. An example of the scale is a casing with width of 10 cm, depth of 8 cm and height of 2.5 cm (with volume of 200 cm³).

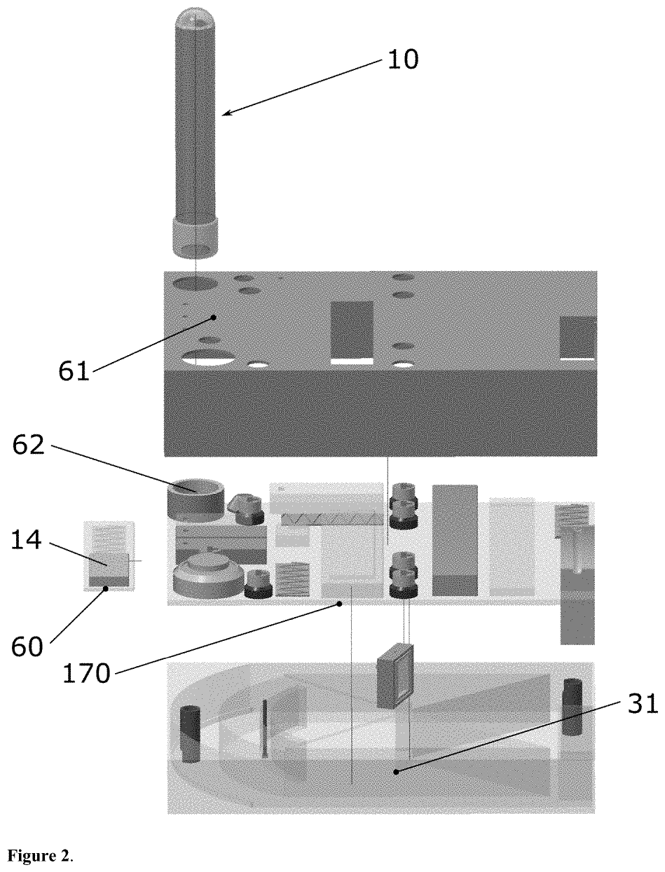

Preferred structures of the cartridge are outlined in FIGS. 2, 3, 21 and 22. Here, socket 62 or 503 can accept a blood collection tube 10. Fluidics compartments 170 and 171 contain the components for the plasma separation and in some embodiments for magnetic bead nucleic acid extraction. In FIG. 2, 31 refers to the electrophoresis gel. After extraction process the nucleic acid sample is transferred to container 14 in a removable part 60 or microtube 502 connected to cartridge casing (e.g. 61 or 65).

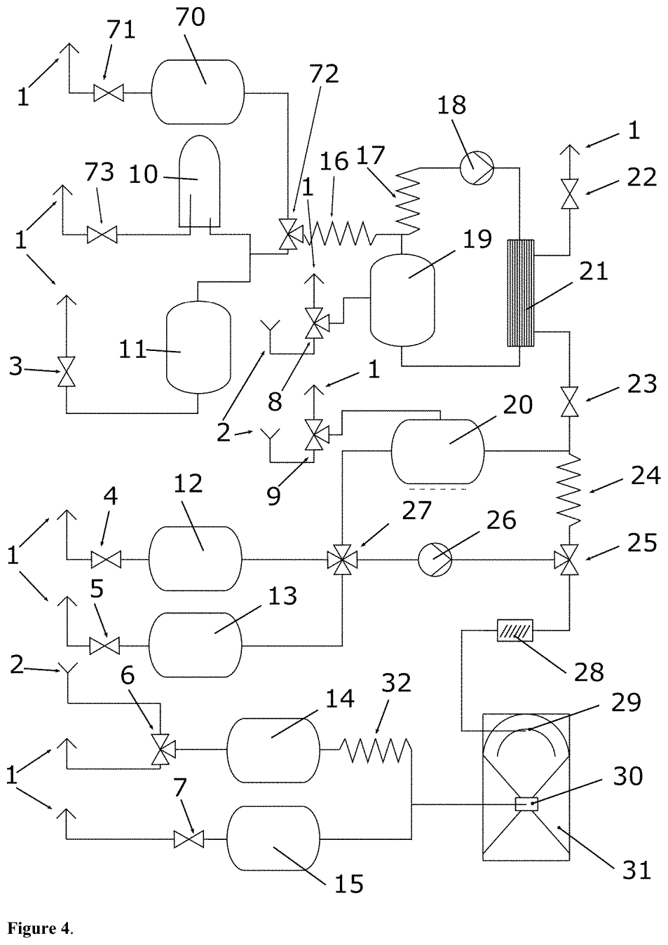

Fluidics of the cartridge preferably include factory-filled, hermetically sealed reagent tanks or containers, analyzers and probes, hollow fiber filter/membrane, mixers, valves, pumps and a site adapted to receive a blood sample tube. For performance, the cartridge requires a power supply and pressure/vacuum provided by an external device. Preferred designs of the fluidics are shown in FIGS. 4-7 and discussed below.

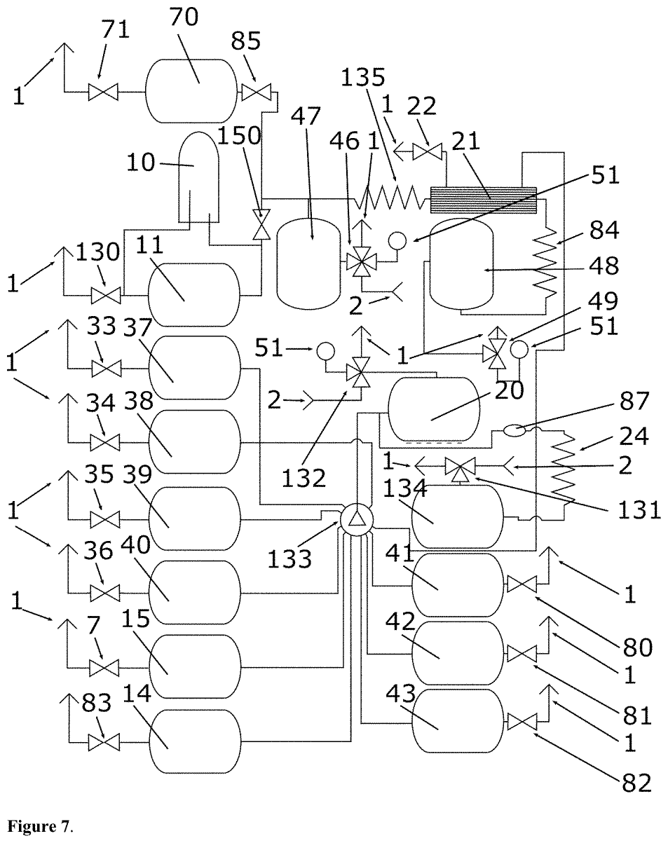

In common for all the designs shown in FIGS. 4-7, connector 1 refers to atmospheric pressure, connector 2 to adjustable vacuum pressure provided by the device and 51 to adjustable positive pressure in form of compressed gas or filtered air. Additionally, in some embodiments the fluidic valves now represented by separate components in the schematics may be implemented as separate components, but in others several valves of the same type may be combined to single multiposition and/or multipath valve. As an example, for schematic in FIG. 7, one implementation could combine following groups into three multiposition valves: a) 7, 22, 33, 34, 35, 36, 46, 49, 71, 80, 81, 82, 130, 131, 132 b) 46, 49, 131, 132 c) 49, 132. This approach can further reduce the cartridge and device footprint and complexity.

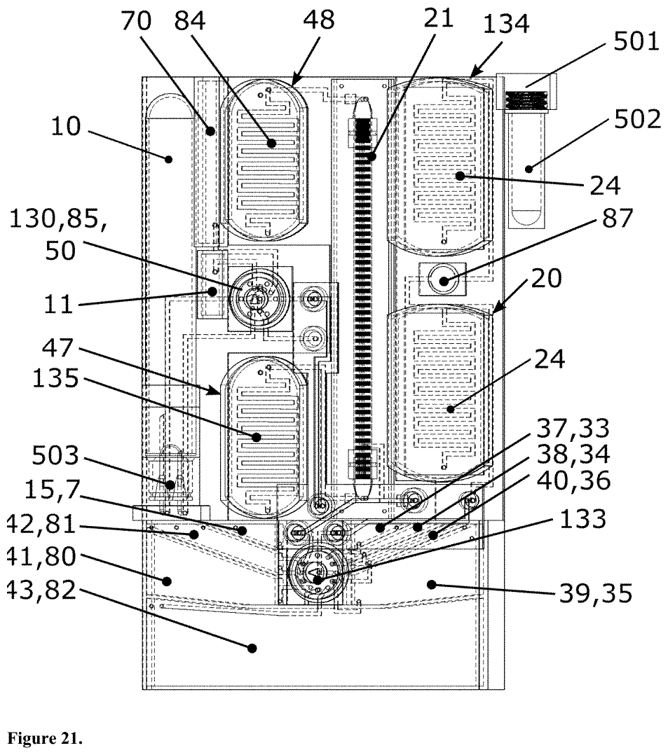

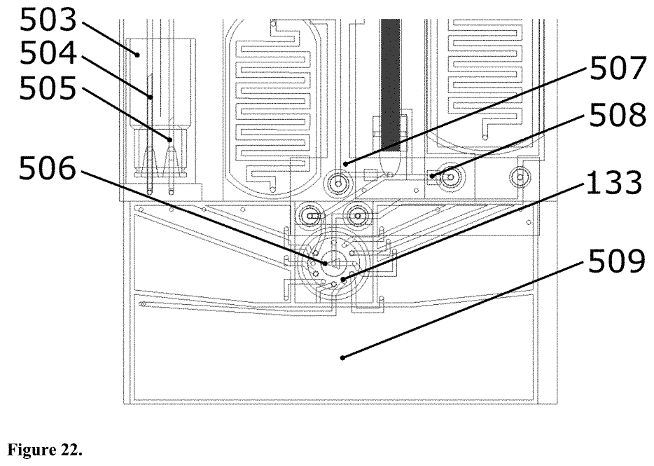

In another configuration (refer to FIGS. 21 and 22), all pneumatic valves (22, 46, 49, 131, 132) can be incorporated into the supporting external device and valves 130, 85 and 50 combined into one multiposition valve. The reagent air valves (7, 33, 34, 35, 36, 71, 80, 81, 82, 83) may be implemented as single-use membrane puncture valves combined into the hermetical sealing mechanism of the reagent tanks (15, 37-43). By using an indexed valve rotor head (506), a reliable interface for operating multiposition valves can be formed between the cartridge and the device.

After insertion of a blood sample tube to the cartridge, the fluidics continues as follows (see FIG. 4): an external device opens the replacement air valves 73 and 3 of the cartridge. Reagent container 11 contains reagent R2 for cell fixation. In the container 19 there is a vacuum through the valve 8, whereby the blood flows from the tube 10 and reagent R2 is added to the blood in the mixer 16 while the blood moves to the blood sample container 19.

Once the entire volume of the sample has been transferred to the 19 (flow can be monitored optically from the duct between the blood tube 10 and the mixer 16), valve 72 is closed and normal atmospheric pressure to the container 19 is returned through valve 8. This is followed by separation of the plasma by using the hollow fiber filter 21. The circulation of the sample of container 19 through filter 21 is facilitated by peristaltic pump 18. Valve 22 is closed and 23 is open to the plasma container 20 with vacuum through valve 9. Thus, when recycled, the plasma released continuously flows into the container 20. Initially, the best filtering conditions were found between 80 and 180 ml/h through an array of 10-15 parallel fibers with external vacuum of the fibers being from 20 to 40 mbar. When the amount of recycled plasma corresponds to 3-4 sample volumes or the filtered cells are sufficiently concentrated (cell density is measured by optical filter from the input of the filter and the plasma quality from the exterior of the output channel), the cells may be rinsed in order to increase the yield and to release vesicles and biomarkers which are more tightly bound to the cells. For this reagent R1 (2-3 ml) in container 70 is used. Reagent R1 can be added while the filtration continues, by opening the valves 71 and 72.

After filtration and rinsing, pump 18 is switched off, valve 22 is opened and the pressure at the vacuum inlet of valve 9 is raised to a level of 100-150 mbar, whereby the flowing air draws the separated plasma into the container 20. When the optical sensor located in the channel between the 21 and 23 confirms that only air flows through the filter, the vacuum in plasma container 20 is released from the valve 9 and the valve 22 is closed. In the next step, the plasma is heated in the container 20 to the temperature required by the pre-treatment reagent (for example, 37-55° C., the cartridge includes an interface to the temperature control section of the external device). Then reagent R3 in container 12 (for example proteinase K) is added by opening the air valve 4 and the valve 27, after which the reagent can be pumped into the plasma by pump 26. Once the reagent has been transferred, valve 27 is set to the position whereby the mixture of plasma and the reagent can be recycled by pump 26 via valve 25 and mixer 24 in a closed loop back into the container. Recycling ensures effective interaction between the reagent and plasma. When pretreatment is complete, reagent R4 in container 13 (e.g., buffer, SDS, markers and other excipients) may be added via the valve 27 by a pump. Once the second reagent treatment (optionally at a different temperature) is completed, the valve 25 is turned to the filter 28 through which the pretreated plasma is pumped and loaded to the electrophoresis gel loading well 29 on the lower compartment of the cartridge.

Upon completion of electrophoresis, the purified and concentrated nucleic acid sample is in the recovery well in the running buffer. From there, it can be collected by making the container 14 depressurized via valve 6 (vacuum 50-200 mbar). However, before the valve 6 is opened, the air valve 7 is also opened, whereby the sample mixes with the stabilizing reagent in the mixing channel 32 to support the preservation of the nucleic acid sample at room temperature (e.g., buffered EDTA, ethanol, propanol, PEG). At the end of the run, the container CD14 containing the nucleic acid sample can be removed from the cartridge.

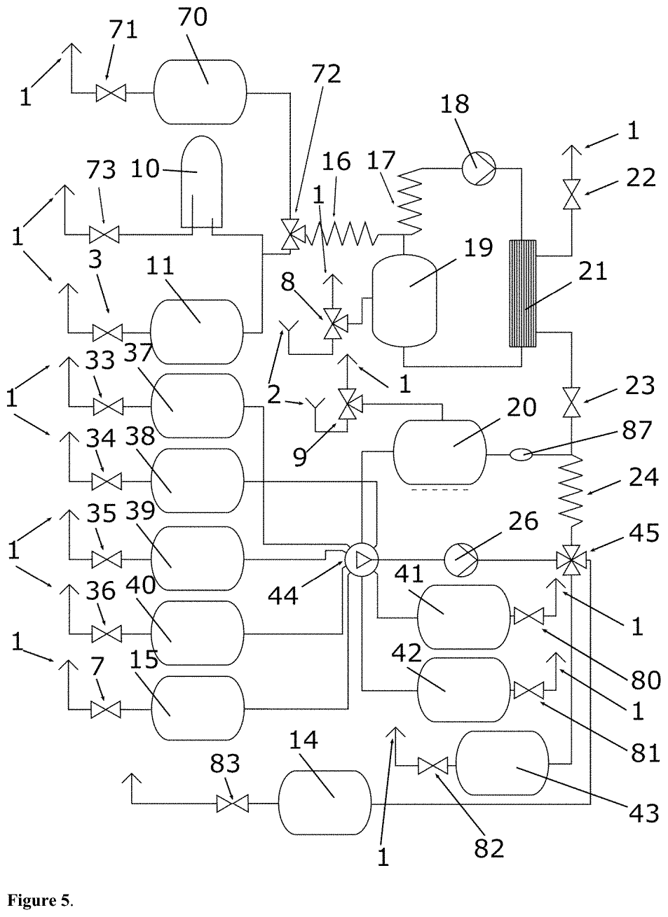

In another embodiment using magnetic bead technology for the nucleic acid extraction, the process takes place according to following steps: After insertion of a blood sample tube to the cartridge, the fluidics continues as follows (see FIG. 5): an external device provides regulated vacuum pressure to connections 2 and opens the replacement air valves 73 and 3 of the cartridge. Reagent container 11 contains reagent R2 for cell fixation. In the container 19 there is a vacuum through the valve 8 and valve 72 is open to tube 10, whereby the blood flows from the tube 10 and reagent R2 is added to the blood stream in the mixer 16 while the blood moves to the sample container 19. Once the entire volume of the sample has been transferred to the container 19 (flow can be monitored optically from the duct between the blood tube 10 and the mixer 16), valve 72 is closed and normal atmospheric pressure to the container 19 is returned through valve 8. This is followed by separation of the plasma using the hollow fiber filter 21. The circulation of the sample in container 19 through filter 21 is facilitated by peristaltic pump 18. Valve 22 is closed and valve 23 is open to the plasma container 20 with vacuum through valve 9. Thus, when recycled, the released plasma flows continuously into the container 20. Initially, the best filtering conditions were found between 80 and 180 ml/h through an array of 10-15 parallel fibers with external vacuum of the fibers being from 20 to 40 mbar. When the amount of recycled plasma corresponds to 3-4 sample volumes or the filtered cells are sufficiently concentrated (cell density is measured by optical filter from the input of the filter and the plasma quality from the exterior of the output channel), the cells may be rinsed in order to increase the yield and to release vesicles and biomarkers which are more tightly bound to the cells. For this reagent R1 (2-3 ml) in container 70 is used. Reagent R1 can be added while the filtration continues, by opening the valves 71 and 72.

After filtration and rinsing, pump 18 is switched off, valve 22 is opened and the pressure at the vacuum inlet of valve 9 is raised to a level of 100-150 mbar, whereby the flowing air draws the separated plasma into the container 20. When the optical sensor located in the channel between the filter 21 and valve 23 confirms that air constantly flows through the filter, the vacuum in plasma container 20 is released from the valve 9 and the valve 22 is closed. In the next step, the plasma is heated in the container 20 to the temperature required by the pre-treatment reagent (for example, 37-55° C., the cartridge includes an interface to the temperature control section of the external device). Then reagent R6 in container 37 (for example proteinase K) is added by opening the air replacement valve 33 and the reagent valve 44, after which the reagent can be pumped into the plasma by pump 26. Additional reagents R7, R8 and R9 (for example lysis buffer, binding buffer and magnetic beads) from reagent containers 38, 39 and 40 are added sequentially with the help of pump 26 by keeping open replacement air valve 9, opening valves 34, 35 and 36 respectively for each reagent and adjusting the valve 44 for relevant flow. Once the reagents have been transferred, valves 44 and 45 are set to the positions whereby the mixture of plasma and the reagent can be circulated by pump 26 via valves 44 and 45 through mixer 24 in a closed loop back into the container. Recirculation ensures effective interaction between the reagent and plasma. When pretreatment and nucleic acid binding steps are complete, contents of reaction container 20 are transferred to waste container 43 with pump 26 by first opening valves 82 and 9 to ambient pressure and then adjusting valve 45. Nucleic acid molecules are bound to the stationary magnetic bead phase, which is retained in the container 20 or the adjacent chamber 87 through magnetic interactions with a permanent magnet or an electromagnet.

After waste removal, valve 45 is changed to direct flow to mixer 24 and wash reagent R10 is introduced from container 41 by opening the air replacement valves 80 and 9 and transferring it with pump 26 through reagent valve 44 into container 20. After introduction of wash reagent, valve 44 is changed back to the enable washing of magnetic beads by circulation between container 20 and mixer 24 and magnetic field is removed to re-suspend the beads into the washing reagent. After wash, beads are again retained in chamber 87 or container 20 with magnetic field and the used wash solution is transferred to waste container 43 through valve 45 with pump 26 after opening air replacement valves 9 and 82. This washing step with reagent R10 can be repeated identically 1-3 times. Then, second wash reagent R11 is introduced into the container 20 by closing valve 45 to waste, opening replacement air valves 81 and 9 and adjusting valve 44 to permit the reagent R11 to flow from container 42 into container 20. After introduction of wash reagent R11, valve 44 is changed back to container 20 to enable washing by circulation between container 20 and mixer 24 and magnetic field is removed to re-suspend the beads into the washing reagent. After washing is complete, beads are retained in chamber 87 or container 20 by applying a magnetic field during circulation of the bead containing solution through the system. Then, wash reagent is transferred to waste container 43 with pump 26 by first opening the valve 45 and then replacement air valves 9 and 82. Washing with second wash reagent R11 can be repeated in identical manner 1-3 times as needed by the purification protocol.

After wash with both reagents R10 and R11 has finished, magnetic beads, which are retained in chamber 87 or container 20 by a magnetic field, are recovered by suspending them into nucleic acid stabilization reagent R12. This solution is transferred to container 20 from container 15 by pump 26 after first opening replacement air replacement valves 7 and 9 and then reagent valve 44. When R12 has been transferred into container 20, valve 44 is adjusted to allow circulation between mixer 24 and container 20 and the removal of magnetic field allows complete re-suspension of the magnetic beads into the solution. Then, replacement air valves 83 and 50 are opened to allow beads suspended in stabilization reagent R12 to flow out of container 20 and into the final reagent container 14. At the end of the run, container 14 containing the nucleic acid sample in a storage reagent R12 can be separated from the rest of the cartridge.

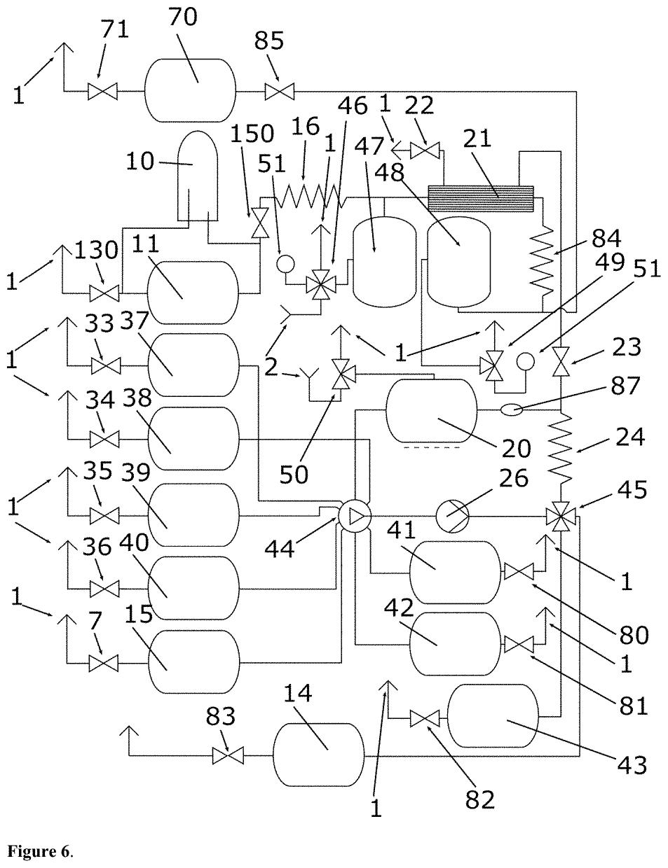

In another embodiment using magnetic beads for nucleic acid extraction, the pump in plasma separation step is replaced with a gas pressure transfer system. Here, the extraction process takes place according to following steps: After insertion of a blood sample tube to the cartridge, the fluidics system operates as follows (see FIG. 6): an external device provides regulated vacuum pressure to connections 2, positive gas pressure to connections 51 and opens the replacement air valve 130 of the cartridge. Reagent container 11 contains reagent R2 for cell fixation. In the container 47 there is a vacuum through the valve 46 and valve 150 is open to tube 10, whereby the blood flows from the tube 10 and reagent R2 is mixed into the flow in the mixer 16 while the blood moves to the sample container 47. Once the entire volume of the sample has been transferred to the container 47 (flow can be monitored optically from the duct between the blood tube 10 and the mixer 16), valve 150 is closed and normal atmospheric pressure to container 47 is returned through valve 46. This is followed by separation of the plasma using the hollow fiber filter 21. Here, valve 22 is closed and 23 is open to the plasma container 20 with vacuum through valve 50. Thus, when recycled, the plasma released flows continuously into container 20. The transfer of the blood from container 47 through filter 21 is facilitated by opening the valve 49 into atmospheric pressure and applying positive gas pressure (initially 150-250 mbar) from regulated source 51 through valve 46 into the container 47. This transfers the blood sample from container 47 through filter 21 into second container 48. The completion of this process can be monitored by detecting the introduction of air into the line between container 47 and filter 21. Once the first transfer of sample over the filter is complete, valve 46 is opened to atmospheric pressure and container 48 is pressurized by letting regulated gas pressure in through valve 49 (second filtration with pressure in the range of 200-380 mbar). This facilitates the second transfer of the blood sample through the fiber filter 21. After completion of this reverse transfer as detected by optical air sensor in-line between container 48 and filter 21, valve 49 is opened to atmospheric pressure and container 47 instantly after pressurized by opening valve 46 to the regulated gas source (third filtration using pressure 300-450 mbar). This sequence results in the third transfer of blood through filter 21 into container 48. Releasing the pressure in container 47 by opening valve 46 into atmosphere stops the flow and allows the addition of cell rinse reagent R1 from container 70 to the container 48 by opening replacement air valve 71, reagent valve 85 and letting vacuum in to container 48 by valve 49. After both remaining blood and washing reagent are in container 48, the valves 85 and 71 are closed, valve 46 is opened to atmosphere and container 48 is pressurized by valve 49 (rinse filtration 260-500 mbar) to drive the fourth circulation of blood through the filter 21.

After filtration and rinsing steps are complete, both chambers 47 and 48 are vented to atmospheric pressure and finally valves 46 and 49 closed to stop any flow of the blood sample. Then valve 22 is opened and the pressure at the vacuum inlet of valve 9 is raised to a level of 100-150 mbar, whereby the flowing air draws the separated plasma into container 20. When the optical sensor located in the channel between the filter 21 and valve 23 confirms constant flow of air through the filter, the vacuum in plasma container 20 is released by valve 9 and valves 22 and 23 are closed. In the next step, the plasma is heated in container 20 to the temperature required for the pre-treatment reagent (for example, 37-55° C., the cartridge includes an interface to the temperature control section of the external device). Then reagent R6 in container 37 (for example proteinase K) is added by opening the replacement air valve 33 and valve 44, after which the reagent can be pumped into the plasma by pump 26. Additional reagents R7, R8 and R9 (for example lysis buffer, binding buffer and magnetic beads) from reagent containers 38, 39 and 40 are added sequentially with the help of pump 26 by opening the replacement air valves 9 and for each reagent valves 34, 35 and 36 respectively and adjusting the valve 44 for relevant flow. Once the reagents have been transferred, valve 44 is set to the position whereby the mixture of plasma and the reagents can be circulated by pump 26 via valve 45 and mixer 24 in a closed loop back into the container. Recirculation ensures effective interaction between the reagent and plasma. When pretreatment and nucleic acid binding steps are complete, contents of reaction container 20 are transferred to waste container 43 with pump 26 by first opening replacement air valves 82 and 50 and then adjusting valve 45. Nucleic acid molecules are bound to the stationary magnetic bead phase, which is retained in the container 20 or the adjacent chamber 87 through magnetic interactions with a permanent magnet or an electromagnet, enabled during the last circulation rounds of the reagent. The length of the trapping process is determined by the reagent solution viscosity and chemical interactions with beads.

After waste removal, valve 45 is changed to direct flow to mixer 24 and wash reagent R10 is introduced from container 41 by opening the air replacement valves 80 and 50 and transferring it with pump 26 through valve 44 into container 20. After introduction of wash reagent, valve 44 is changed back to the enable washing by circulation between container 20 and mixer 24 and magnetic field is removed to re-suspend the beads into the washing reagent. After wash, beads are again retained in container 20 or chamber 87 with magnetic field and the solution is transferred to waste container 43 through valve 45 with pump 26 after opening replacement air valves 82 and 50. This washing step with reagent R10 can be repeated in identical manner 1-3 times. Then, second wash reagent R11 is introduced into the container 20 with pump 26 by opening replacement air valves 81 and 50 and adjusting valve 44 to permit reagent R11 flow from container 42 into container 20. After introduction of wash 2 reagent R11, valve 44 is changed back to container 20 to enable washing by circulation between container 20 and mixer 24 and magnetic field is removed to re-suspend the beads into the washing reagent. After wash completes, beads again are retained in container 20 or chamber 87 by applying a magnetic field during circulation of the bead solution. Then, wash reagent is transferred to waste container 43 with pump 26 by first opening the valves 45, 82 and 50. Washing with second wash reagent R11 can be repeated in identical manner 1-3 times as needed by the purification protocol.

After wash with both reagents R10 and R11 has finished, magnetic beads, which are retained in container 20 or chamber 87 by magnetic field are now recovered by suspending them into nucleic acid stabilization reagent R12 which is transferred in from container 15 by pump 26 after first opening replacement air valve 7 and 50 and then reagent valve 44. When R12 is in container 20, valve 44 is adjusted to allow circulation between mixer 24 and container 20, where the removal of magnetic field allows complete re-suspension of the magnetic beads into the solution. Then, replacement air valves 83 and 50 are opened to allow beads in stabilization reagent R12 to flow out of container 20 and into the final reagent container 14. At the end of the run, the container 14 containing the nucleic acid sample in a storage reagent R12 can be separated from the rest of the cartridge.

In another embodiment using magnetic beads for the nucleic acid extraction, the fluidic operations are performed entirely using vacuum and gas pressure. Here, the extraction process takes place according to following steps: After insertion of a blood sample tube to the cartridge, the fluidics system operates as follows (see FIG. 7): an external device provides regulated vacuum pressure to connections 2, positive gas pressure to connections 51 and opens the replacement air valve 130 of the cartridge. Reagent container 11 contains reagent R2 for cell fixation. In the container 47 there is a vacuum through the valve 46 and valve 150 is open to tube 10, whereby the blood flows from the tube 10 and reagent R2 is added to the stream in the mixer 16 while the blood moves to the sample container 47. Once the entire volume of the sample has been transferred to container 47 (flow can be monitored optically from the duct between the blood tube 10 and the container 47), valve 150 is closed and normal atmospheric pressure to container 47 is returned through valve 46. This is followed by separation of the plasma using the hollow fiber filter 21. Here, valve 22 is closed and 133 is open to the plasma container 20 with vacuum through valve 132. Thus, when recycled, the released plasma flows continuously into container 20. The transfer of the blood from container 47 through mixers 135, 84 and filter 21 is facilitated by opening the valve 49 into atmospheric pressure and applying positive gas pressure (initially 150-250 mbar) from regulated source 51 through valve 46 into the container 47. This transfers the blood sample from container 47 through filter 21 into second container 48. The completion of this process can be monitored by detecting the introduction of air into the line between container 47 and filter 21. Once the first transfer of sample over the filter is complete, valve 46 is opened to atmospheric pressure and container 48 is pressurized by letting regulated gas pressure in through valve 49 (second filtration with pressure in the range of 200-380 mbar). This drives the second transfer of the blood sample through the fiber filter 21. After completion of reverse transfer as detected by optical air sensor in-line between container 48 and filter 21, valve 49 is opened to atmospheric pressure and container 47 is instantly afterwards pressurized by opening valve 46 to the regulated gas source (third filtration using pressure 300-500 mbar). This sequence results in the third transfer of blood through filter 21 into container 48, which is then followed by another transfer in the earlier described sequence from container 48 through filter 21 again to container 47 (fourth filtration using pressure 400-550 mbar). Releasing the pressure in container 48 by opening valve 49 into atmosphere stops the flow and allows the addition of cell rinse reagent R1 from container 70 to the container 47 by opening the replacement air valve 71, reagent valve 85 and letting vacuum into the container 47 by valve 46. After both remaining blood and washing reagent are in container 47, the valve 85 is closed and container 47 pressurized by valve 46 (rinse filtration pressure 250-500 mbar) to drive the fourth circulation of blood through the filter 21.

After filtration and rinsing steps are complete, both chambers 47 and 48 are vented to atmospheric pressure and finally valves 46 and 49 closed to stop any flow of the blood sample. Then valve 22 is opened and the pressure at the vacuum inlet of valve 9 is raised to a level of 100-150 mbar, whereby the air flowing in from valve 22 draws the separated plasma into container 20. When the optical sensor located in the channel between the filter 21 and valve 133 confirms constant flow of air through the filter 21 on filtrate side, the vacuum in plasma container 20 is released by valve 132 and valve 22 and 133 are closed. In the next step, the plasma is heated in container 20 to the temperature required for the pre-treatment reagent (for example, 37-55° C., the cartridge includes an interface to the temperature control section of the external device). Then, reagent R6 from container 37 (for example proteinase K) is added by opening the replacement air valve 33 and valve 132 into vacuum, after which the reagent is transferred to the plasma in container 20 by opening the reagent valve 133 for a predefined time. Additional reagents R7, R8 and R9 (for example lysis buffer, binding buffer and magnetic beads) from reagent containers 38, 39 and 40 are added sequentially in identical manner by opening replacement air valves 34, 35 and 36 respectively, for each reagent, letting vacuum into reaction container 20 through valve 132 and adjusting the valve 133 for the relevant reagent flow. Once the reagents have been transferred, valve 133 is kept closed and mixture of plasma and the reagent is circulated between the containers 20 and 134 through mixer 24. This is achieved by letting atmospheric pressure to container 20 through valve 132, opening valve 45 and taking container 134 to vacuum pressure through valve 131. The reverse transfer is performed by opening container 134 to atmospheric pressure through valve 131 and letting vacuum into the container 20 with valve 132. Mixing can be continued as long as necessitated in protocol by repeating the transfer steps. After the last transfer, sample will remain in the container 20. When pretreatment and nucleic acid binding steps are complete, contents of reaction container 20 are transferred to waste container 43 with gas pressure by first opening replacement air valve 82, reagent valve 133 and then adjusting container 20 to positive gas pressure by valve 132. Nucleic acid molecules are bound to the stationary magnetic bead phase, which is retained in the container 20 or the adjacent chamber 87 through magnetic interactions with a permanent magnet or an electromagnet.

After waste removal, valve 133 is changed to direct flow to mixer 24 and wash reagent R10 is introduced from container 41 by opening the replacement air valve 80, taking container 20 to vacuum pressure by valve 132 and opening reagent valve 133 for a predefined time. After introduction of wash reagent, valve 133 is closed again and magnetic field is removed to re-suspend the beads into the washing reagent. Washing takes place by circulating solution between the containers 20 and 134 through mixer 24. This is achieved by letting atmospheric pressure to container 20 through valve 132, opening valve 45 and taking container 134 to vacuum pressure through valve 131. The reverse transfer is performed by opening container 134 to atmospheric pressure through valve 131 and letting vacuum into the container 20 with valve 132. Washing can be continued as long as necessary by repeating the two transfer steps in succession. During the last transfer cycles, beads are collected in container 20 or chamber 87 by applying a magnetic field. After the last transfer, solution remains in the container 20.

After wash, beads are retained in the magnetic field and the wash solution is transferred to waste container 43 by opening reagent valve 133, replacement air valve 82 and letting gas pressure into container 20 from valve 132. This washing step with reagent R10 can be repeated in identical manner 1-3 times. Then, second wash reagent R11 is introduced into the container 20 by opening the air replacement valve 81, taking container 20 to vacuum pressure with valve 132 and opening reagent valve 133 to container 42 for a predefined time. After introduction of wash 2 reagent R11, valve 133 is closed and magnetic field is removed to re-suspend the beads into the washing reagent. Washing takes place by circulating solution between the containers 20 and 134 through mixer 24. This is achieved by letting atmospheric pressure into container 20 through valve 132, opening valve 45 and adjusting container 134 to vacuum pressure through valve 131. The reverse transfer is performed by opening container 134 to atmospheric pressure through valve 131 and letting vacuum into the container 20 with valve 132. Washing can be continued as long as necessary by repeating the transfer steps. During the last transfer cycles, beads are collected in container 20 or chamber 87 by applying a magnetic field. After the last transfer, solution remains in the container 20.

After washing, solution from reaction container 20 is transferred to waste container 43 with gas pressure by first opening replacement air valve 82, reagent valve 133 and then adjusting container 20 to positive gas pressure with valve 132. Washing with second wash reagent R11 can be repeated in identical manner 1-3 times as needed by the purification protocol.

After wash with both reagents R10 and R11 has finished, magnetic beads, which are retained in container 20 or chamber 87 by magnetic field are recovered by suspending them into nucleic acid stabilization reagent R12. This solution is transferred in from container 15 by opening replacement air valve 7, letting vacuum to container 20 by valve 132 and opening reagent valve 133 for a predefined time. When R12 is in the container 20, valve 133 is closed and valve 45 opened to allow sample transfer between containers 20 and 134 by regulating the under- and overpressure by the valves 131 and 132. When magnetic field has been removed, beads are suspended to the solution as it flows from container 20 to container 134 by opening valve 131 into vacuum and valve 132 to the atmospheric pressure. When storage solution with the beads is in the container 134, it is again transferred back to container 20 by opening valve 132 to vacuum and valve 131 to atmospheric pressure. In the last step, bead solution is transferred to the final sample container 14 by opening the replacement air valve 83, reagent valve 133 and valve 132 to positive gas pressure. After the end of the run, the container 14 containing the nucleic acid sample in a storage reagent R12 can be separated from the rest of the cartridge. This container can take the form of a regular microtube or a more advanced design combining additional features as described in FIG. 14.

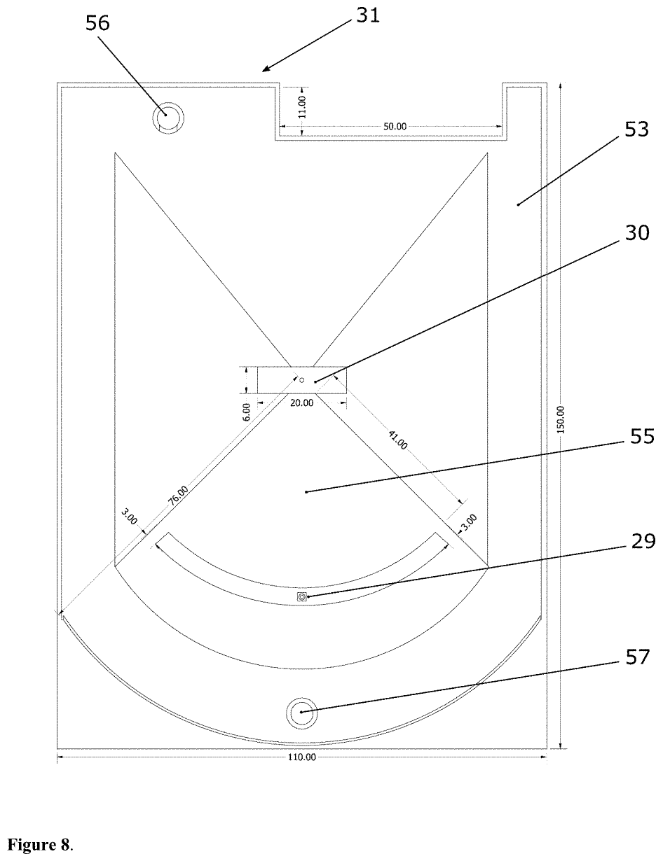

In a preferred embodiment, the form of the electrophoresis gel (55) in the running chamber (31) corresponds to a sector of a circle (see FIG. 8) so that the wider end comprises a loading well (29) for a 5-7 ml plasma sample to be purified and concentrated in the gel. The sector form produces a heterogeneous electric field for the gel and compensates for the laminar flow pattern of the molecules, whereby the migrating zones retain their shape and size separation is possible. Similar effect is not readily achievable, for example, with a rectangular or triangular gel, because the shape of the migrating zones extends significantly, limiting the size separation and yield.

Preferably, the tip of the gel sector is truncated (see FIG. 8) so that the gel ends up in a collecting vessel (30) where there is a space between the two filters filled with the running buffer, having a volume of 300-600 μl. On the gel side of the collecting vessel, there is a filter (for example, Whatman Durapore, 0.45 μm, PVDF) through which the nucleic acid molecules can pass. On its opposite side, there is another filter that prevents nucleic acids from passing, but penetrates the small-sized ions contained in the running buffer. Such filter materials include, for example, a 15 nm filter (Whatman Nuclepore, 0.015 μm, PC), PVDF ultrafiltration membranes in the size selection range 20-70 kDa or ion exchange membranes (e.g., Sustainion, X37-50).

The average distance traveled by the nucleic acids in the electrophoresis gel is about 40 mm, preferred gel agarose concentration is 0.5-1%, preferred gel volume is 50-100 ml, and preferred running buffer volume about 200 ml. Over the gel, a voltage of 150-250 V (10-17 V/cm) is preferably maintained and the running time is 30 to 60 minutes or less. During electrophoresis, the running buffer (53) is recycled between the ends of the gel to prevent pH change. To achieve this, peristaltic pump is used to transfer liquid between orifices 56 and 57, for this the pump has a second hose next to the sample channel to recycle the running buffer. The buffer flow rate can be 10-30 ml/min. Suitable active running buffer ingredients include borate, phosphate and glycine at concentrations of 5-25 mM. Primarily, 10-20 mM borate buffer has been used in the tests. The recycled buffer or the gel can also be cooled during electrophoresis, whereby the maintained voltage can be raised so that the running time is reduced.

The migration of nucleic acids in the gel is monitored by an optical sensor that measures fluorescence or absorbance through the gel in front of the collecting vessel. When the run is complete, the polarity of the gel is reversed for 2 to 200 seconds, whereby the nucleic acid molecules are removed from the latter filter of the collecting vessel to the running buffer in the collecting vessel wherefrom the nucleic acid sample is pumped out of the gel.

In the particular embodiments, the present invention is thus directed to a cartridge for extraction of circulating nucleic acids from a blood sample, the cartridge comprising a first compartment for filtering plasma from a blood sample in order to prepare plasma which is free of cells and has a minimal amount of cellular nucleic acids. Preferably, the plasma is obtained by fixing the cells in the first compartment prior to the filtering to minimize the leakage of cellular nucleic acids. The cartridge also comprises a second compartment for performing nucleic acid extraction, wherein the first compartment comprises a hollow fiber filter and the second compartment comprises a chamber for nucleic acid extraction and solid-phase binder material such as magnetic beads. Preferably, said first compartment also comprises means to receive a blood sample. In said first compartment, the cartridge preferably comprises means to mix said blood sample with a cell fixation reagent encapsulated in said first compartment.

In another preferred embodiment, said first compartment comprises means to mix a plasma sample separated from said blood sample in a hollow fiber membrane with an enzymatic or chemical reagent encapsulated in said first compartment.

In another preferred embodiment, said first compartment comprises means to transfer the separated plasma mixed with said enzymatic or chemical reagent from the first compartment to the second compartment.

In another preferred embodiment, said cartridge, more preferably the second compartment, comprises a preservative or a stabilizing agent encapsulated in said cartridge, more preferably in said second compartment, means to mix said preservative or a stabilizing agent with nucleic acid fragments bound to said solid-phase binder material and a removable container for storing the mix of the nucleic acid fragments and said preservative or a stabilizing agent.

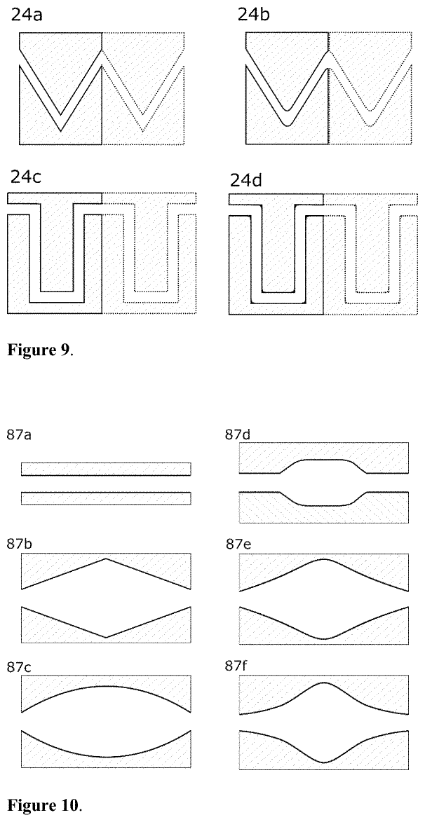

Preferably, the means of mixing include low-cost passive fluidic structures not requiring any mechanical actuation, but rather take advantage of the turbulent-like flow properties of liquid-solid suspensions in patterned channels. FIG. 9 shows alternative fluidic channel top view configurations (24a-d) which can be used to decrease the required flow rate for overcoming the critical Reynolds number (Re) for reagent solutions and enabling efficient turbulent mixing in fluids of medium to high viscosity. Of these, configurations 24c and 24d provide the lowest critical Re, but their shape can decrease the removal efficiency of liquid and solid reagents from the channel and thus structure is 24c preferred in this regard. Configurations 24a and 24b provide a compromise in terms of ease of removing existing reagents, but require a higher rate for efficient mixing.

Additionally, dynamic mechanical mixing can be used. One preferred configuration using a rotating stirrer blade is shown in FIG. 12. Here, blade 114 can be rotated in a closed liquid chamber 20a (refer to FIGS. 4-7) formed by joining top piece 112 and bottom piece 116. Liquid can move through orifices 117 and 118 and pneumatic pressure through connection 113. Chamber is sealed from the environment by an elastomer rotor seal disc 111 which is compressed against the chamber top element by retaining plate 110 to provide an enclosed system. Circular inner surface of the seal 111 functions as the top bearing for the blade 114, allowing it to rotate inside the container 20a. Bottom end of the shaft holding the blade 114 has a spherical overhang to functions as a second bearing in spherical recess 115 at the bottom of the chamber 20a. Blade rotation is provided by a mechanical connection to an actuator in the device through upper part of shaft 118.

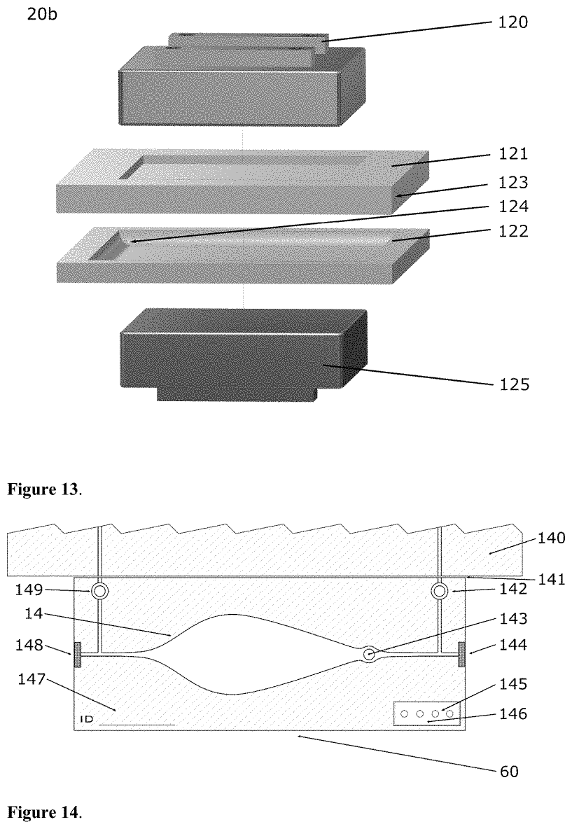

Alternatively, dynamic mixing can be provided by means of manipulating the solid phase material suspended in the liquid reagents. Especially suitable for this application are paramagnetic particles, which can also include coating layer to function as a solid-phase reversible binder for nucleic acids. FIG. 13 demonstrates a preferred mixer configuration 20b (refer to FIGS. 4-7), where permanent magnets or electromagnets 120 and 125 are used to manipulate the particle containing liquid phase inside the container 20b formed by plates 121 and 122 where the liquid can flow through connections 123 and 124. In operation with permanent magnets 120 and 125, one of the magnets at time is brought into the outside recess on the top or bottom plates 121 and 122, which draws the particles in the solution to the magnet side of the chamber 20b. To perform efficient mixing, the movement step is repeated several times, alternating the magnets 120 and 125 while simultaneously removing the other magnet from the chamber. With electromagnets, similar action can be performed by alternating the magnet to which the electric current is applied to. Based on the dynamic properties of the liquid and the magnetic particles, cycle rate of the mixing action can be adjusted to enable efficient material transfer during each cycle. Interaction with the plate magnet 120 or 125 also comprises efficient means for retaining the magnetic particles in the container 20b to enable replacement of the liquid phase.

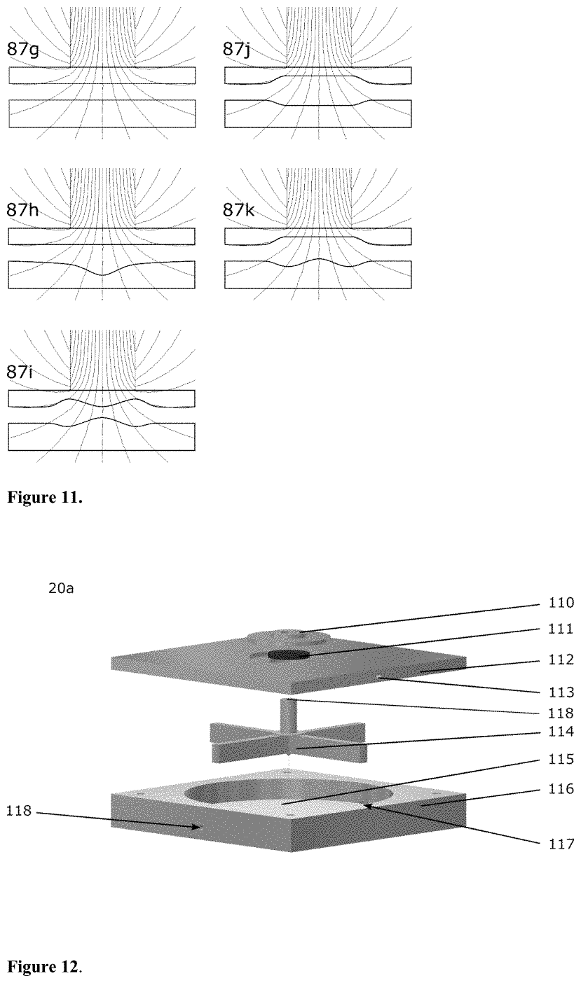

In one preferred configuration, the cartridge can also include a fluidic fixture for capturing and retaining magnetic particles to enable the replacement of the suspending phase. FIG. 10 shows alternative top view configurations (a-f) for the bead capture fixture 87 (refer to FIGS. 5-7) and FIG. 11 shows alternative side view configurations (g-k) for the fixture 87. FIG. 11 also includes a partial view of the magnetic field lines defining the preferred relative orientation of the magnetic pole. Any pair of the top and side view configurations from FIGS. 10 and 11 can be combined with each other to form efficient capture geometries for fluid reagent and particles with different dynamic properties. Of these configurations in FIG. 11, 87j is the most applicable for flows of high Reynolds number and 87k, 87i, 87h and 87g are applicable for flows with decreasing Re in this order. Similarly in FIG. 10, configuration 87c is more suitable for high Re flows and configurations 87e, 87f, 87b, 87d and 87a are adjusted for low Re flow conditions. The specified capture geometries can be used in conjunction with electromagnets or permanent magnets with mechanisms to actuate their distance to the fixture. Release of the trapped material after removal of magnetic field can be enhanced with increased flow rate and/or by repeatedly changing the flow direction through the fixture.

In the preferred embodiment of the cartridge, there is as socket for directly interfacing with a collection tube containing the sample material (503 in FIGS. 21 and 22). In more detail (refer to FIG. 22) the socket contains needles 504 and 505 to puncture the sample tube septum seal to enable the outflow of sample and at the same time a controlled inflow of air to compensate for the pressure change inside the tube.

The fluidic channels in the cartridge may be of any appropriate design and can be arranged in one or more interconnected layers. As shown in FIG. 22 two separate, stacked layers of material (507 and 509) may be used to form the channels to facilitate a high-density design and low-cost manufacturing of the fluidic system. By incorporating materials with partial optical transparency and positioning suitable cutouts in the cartridge external casing (508), the fluidic process may be observed by measuring sample absorbance in the channel to enable precise process control. Alternatively, contactless capacitive electronic sensing may be used to measure the current volume of sample in any container or tank (e.g., 20, 47, 48, 134) to infer the current process state.

In another preferred embodiment, the cartridge, more preferably the second compartment, comprises a nucleic acid specific dye encapsulated in said cartridge, more preferably in said second compartment, and means to mix said dye with the nucleic acid fragments bound to said solid-phase binder material or washed from said binder material. Preferably, said nucleic acid specific dye is a double-stranded DNA specific dye. In this embodiment, the cartridge and/or said removable container, described in FIG. 14, preferably comprises a transparent window (143) arranged to direct a beam of light through the sample or mix to measure the amount of isolated nucleic acids bound to said dye. The removable container may also take the form of a regular laboratory microtube (502) in a suitable connector interface (501) as demonstrated in the cartridge configuration in FIG. 21. Here, the fluorescent measurement may be performed in identical manner at the magnetic bead retaining area (87), which is equipped with a transparent window on one side.

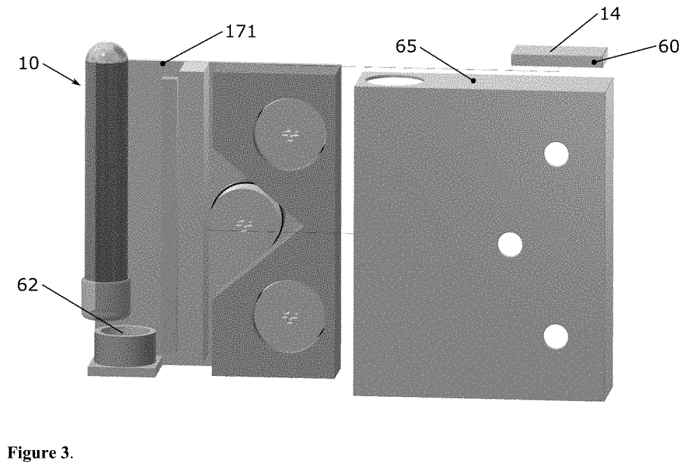

In more detail, in accordance with one embodiment of the invention, the removable sample transfer container 60 (refer to FIG. 14) consists of two normally open, single-action valves at inlet (142) and outlet (149), a container 14 for storing the liquid sample and two needle septums (144 and 148) for recovering the sample from the container for analysis. In addition, for recording sample information the sample container has a writable digital memory chip 146 with electrical connector surfaces 145 to interface with the device, a pen writing area 147 for manual sample information and is separable from the cartridge outer casing (140) along the break-away surface 141. Here, 140 can in some embodiments refer to parts 61 or 170 (FIG. 2) or 65 or 171 (FIG. 3).

After the sample is processed in the cartridge, it is transferred into the container 14 through valve 142 and over the measurement area 143. In one embodiment the sample is in liquid form and extracted nucleic acid concentration is measured during the sample transfer flow to the storage container. In another embodiment the sample is still adsorbed to magnetic beads and a magnet is positioned by the processing device above the collection area 143 or 87 and beads in the flow are collected to the area. Then, a fluorescence intensity measurement can be performed.

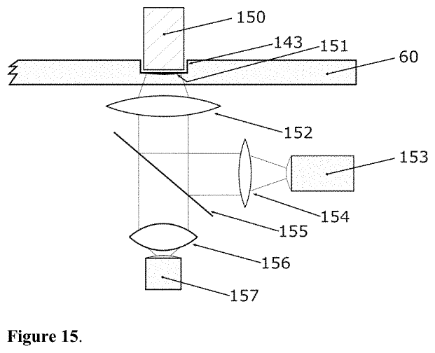

Here according to the configuration shown in FIG. 15, magnet 150 holds the beads (151) in the measurement area 143. In case of a liquid phase sample, 151 refers to the volume in the measurement fixture area 143. Then objective lens 152 transfers the excitation light beam from source 157 through the collimator lens 156 and dichroic splitter mirror 155 to the area 143. The resulting sample fluorescence is converted to electrical signal by photodetector 153 to which the light is collected by focusing lens 154. Suitable excitation sources include light emitting diodes (LED), laser sources, excimer lamps and gas discharge bulbs. Emission photons can be collected with photodiodes, phototransistors, single photon avalanche detectors, charge coupled devices (CCD), complementary metal oxide semiconductor detectors (CMOS) or photomultiplier tubes (PMT).

The present invention is further directed to an automated system for extraction of nucleic acid fragments from a blood sample comprising a device with a docking site or slot adapted to receive the cartridge as defined in any of the above embodiments, said device comprising means adapted to operate the blood plasma filtering process in said cartridge and a) means adapted to operate nucleic acid purification in said cartridge with a binder material specific to nucleic acids or b) means adapted to operate gel electrophoresis in said cartridge. Automated systems supplying and removing buffer or other reagents to/from a cassette or cartridge, as well as the controlled application of electrical voltages for electrophoresis are described, e.g., in US20040050699.

A person skilled in the art would also understand that the principles of the present invention can be applied to purification and storage of protein biomarkers from blood samples. Accordingly, the present invention is also directed to a method for extraction of protein biomarkers from a blood sample, the method comprising the steps of:

a) providing a blood sample taken from an individual;

b) preferably stabilizing blood cells in the sample with a fixative reagent;

c) filtering the blood sample in order to separate plasma from said blood sample by using a hollow fiber filter;

d) preferably washing the separated cell fraction with a washing reagent and collecting the washout;

e) contacting the plasma sample obtained in step c), and preferably the washout collected in step d), with a binder material specific to a protein biomarker in order to purify said biomarker whenever present in said plasma sample;

f) collecting those proteins bound to said binder material; and

g) mixing the proteins collected in step f) with a preservative or a stabilizing agent;

wherein the steps b) to g) are performed on an automated system on a cartridge comprising a first compartment for filtering plasma from a blood sample and a second compartment for contacting the plasma sample obtained in step c) with a binder material specific to said protein biomarker.

In an embodiment, the present invention is directed to a cartridge comprising a first compartment for filtering plasma from a blood sample and a second compartment for contacting a plasma sample with a binder material specific to a protein biomarker, wherein the first compartment comprises a hollow fiber filter and the second compartment comprises a chamber for protein purification and a binder material specific to said protein biomarker encapsulated in said cartridge, wherein said binder material preferably comprises magnetic beads. In a more preferred embodiment, said magnetic beads are coated with an antibody specific for said protein biomarker.

In an embodiment, the present invention is directed to an automated system for extraction of nucleic acid fragments from a blood sample comprising a device with a docking site adapted to receive the cartridge as defined above comprising binder material specific to a protein biomarker, said device comprising means adapted to operate the blood plasma filtering process in said cartridge and means adapted to operate protein purification in said cartridge with a binder material specific to said protein biomarker.

The publications and other materials used herein to illuminate the background of the invention, and in particular, to provide additional details with respect to its practice, are incorporated herein by reference. The present invention is further described in the following experimental section, which is not intended to limit the scope of the invention.

### EXPERIMENTAL SECTION

### Example 1

Comparison of Plasma Prepared with Hollow Fiber Filtration in Different Pumping Modes

Blood was collected from two individuals into K₂EDTA blood collection tubes and stored at 21° C. for a maximum of four hours. Plasma separation was performed from whole blood by passing the sample through a set of polypropylene (average pore size <500 nm) hollow fiber filters with a combined filter surface area of 1800 mm². Fibers were arranged into sets of 10 in parallel with total length of fiber being 1700 mm. Pressure in the plasma compartment outside fibers was −40 mbar relative to atmosphere. Whole blood sample (8-9 ml) was passed through the filter one or three times and the separated plasma collected. Reference separation was performed by centrifugation (2000 g, 10 min, 21° C.). Plasma quality was assessed by measuring the absorbance value (for HbO₂ in EDTA at 576 nm) and comparing it to the centrifugation samples. Blood flow rate was 120 ml/h for peristaltic, syringe and gas pressure pumps (positive pressure of 240-500 mbar depending on the filtration cycle number). Average yield of separated plasma was 47% of blood volume with centrifugation, 16% with syringe pump (single cycle, non-comparable), 41% with peristaltic pump and 42% with gas pressure pumping, excluding the volume of any subsequent rinse reagent used to recover DNA remaining in the flow-through sample intracellular space. Average plasma A₅₇₆ values were 0.890, 1.283, 2.970 and 2.242 for centrifugation, syringe pump, peristaltic pump and gas pressure pump, respectively.

### Example 2

Separation of Plasma from Anticoagulant Treated Whole Blood Using Fiber Filtration for the Extraction of cfDNA

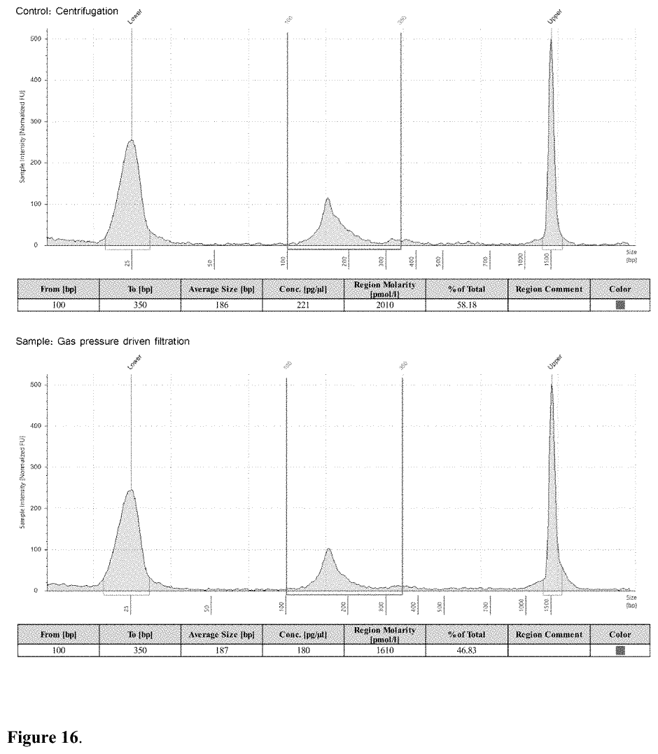

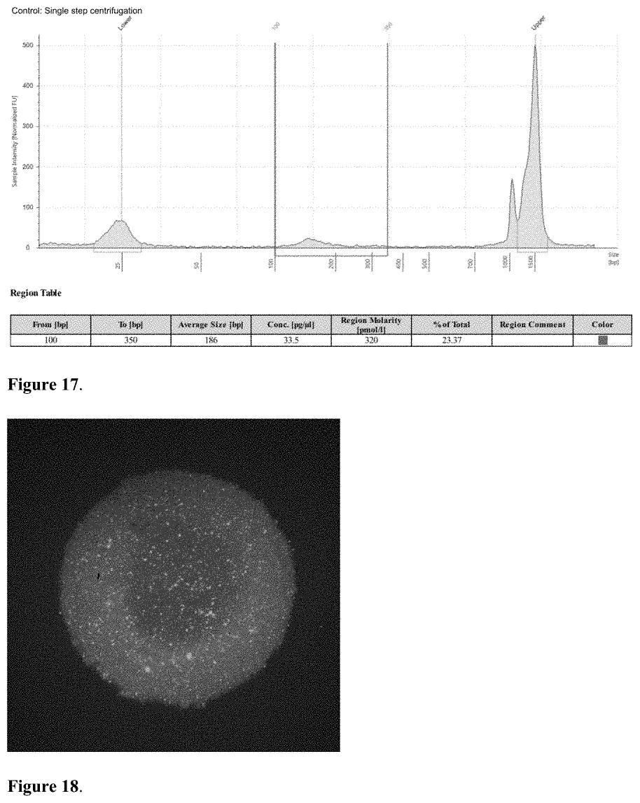

Blood was collected from two individuals into K₂EDTA blood collection tubes and stored at 21° C. for a maximum of four hours. Plasma separation was performed from whole blood by passing the sample through a set of polypropylene (average pore size <500 nm) hollow fiber filters with a combined filter surface area of 1800 mm². Fibers were arranged into sets of 10 in parallel with total length of fiber being 1700 mm. Pressure in the plasma compartment outside fibers was −40 mbar relative to atmosphere. Whole blood sample (8-9 ml) was passed through the filter three times and the separated plasma collected. Reference separation was performed by centrifugation (two-step, 1900 g, 10 min and 16000 g, 10 min, at 21° C. to compare cfDNA quality to single-step centrifugation, 2000 g, 10 min, 21° C.). Blood flow rate was 120 ml/h for peristaltic and gas pressure pumps (positive pressure of 240-500 mbar depending on the filtration cycle number). Cell-free DNA was isolated from the prepared plasma samples using a commercial extraction kit (Perkin Elmer NextPrep-Mag) at 2.5 ml sample scale. Average yield of cfDNA in size range 100-350 base pairs was 4.50, 2.34 (52%) and 3.60 (80%) ng/ml (relative to plasma volume) respectively for centrifugation, peristaltic pump filtration and gas pressure actuated filtration. Representative size fractioning for extracted DNA in a filtration sample and a centrifugation reference is shown in electropherograms in FIG. 16, where peaks marked lower and upper are instrument controls. Here, identical size distribution and similar concentration between treatments confirm that suggested filtration method is able to produce plasma of comparable quality to that recovered by two-step centrifugation for cfDNA extractions. Additionally, cfDNA extracted from plasma obtained with a single-step centrifugation (2000 g, 10 min, 21° C.) is shown as a reference in electropherogram in FIG. 17, where peaks marked lower and upper are instrument controls. In this plot DNA concentration between 100-350 base pairs has significantly reduced and additional fragments are apparent at around 1100 base pairs. This demonstrates the need for a two-step centrifugation protocol to prevent the carryover of leukocytes that can, depending on the extraction protocol, lead to genomic DNA contamination and loss of the original cell free nucleic acids.

### Example 3

Extraction of cfDNA Blood Plasma Using Magnetic Beads in Dynamic Mixing System

Blood plasma was prepared by centrifugation (two-step, 1900 g, 10 min and 16000 g, 10 min, both at 21° C.) and used fresh or stored at −20° C. for short-term or −80° C. long-term. The nucleic acid extraction protocol included an 8 minute lysis step (3 ml plasma, 100 μl lysis buffer at 53° C., 30 μl proteinase K (>600 U/ml)) and a binding step (1.5× ratio of binding buffer to sample, 40 μl highly carboxylated paramagnetic beads) for 10 minutes. First wash with 1.5 ml of W1 reagent was performed twice for 4 minutes and second wash with 1 ml of W2 reagent was performed twice for 2 minutes. Collection of the beads from the mixing system was done with W2 solution. Elution was performed outside the fluidic system. Composition of solutions used was following: Lysis buffer: 10% SDS in water, Binding buffer: 3.5 M NaCl, 15 mM Tris-HCl, 2 mM EDTA, 35% PEG 6000, 0.02% Tween 20 at pH 8.5, W1: 30% guanidine hydrochloride in 60% ethanol, W2: 80 ethanol in water, Elution: water. Mixing and extraction fluidic system consisted of reagent valves, pump and a magnetic mixing fixture (FIG. 13, system 20b). Extraction yield was compared to the same extraction chemistry with manual sample processing. For blood plasma, the average yields were 1.60 and 1.24 (78%) ng/ml relative to plasma for manual and fluidic extraction respectively.

### Example 4

Extraction of cfDNA from Blood Plasma Using Magnetic Beads in Passive Mixing System

Blood plasma was prepared by centrifugation (two-step, 1900 g, 10 min and 16000 g, 10 min, both at 21° C.) and used fresh or stored at −20° C. for short-term or −80° C. long-term. The nucleic acid extraction protocol included an 8 minute lysis step (3 ml plasma, 100 μl lysis buffer at 53° C., 30 μl proteinase K (>600 U/ml)) and a binding step (1.5× ratio of binding buffer to sample, 40 μl highly carboxylated paramagnetic beads) for 10 minutes. First wash with 1.5 ml of W1 reagent was performed twice for 2 minutes and second wash with 1 ml of W2 reagent was performed twice for 4 minutes. Collection of the beads from the mixing system was done with W2 solution. Elution was performed outside the fluidic system. Composition of solutions used was following: Lysis buffer: 10% SDS in water, Binding buffer: 3.5 M NaCl, 15 mM Tris-HCl, 2 mM EDTA, 35% PEG 6000, 0.02% Tween 20 at pH 8.5, W1: 30% guanidine hydrochloride in 60% ethanol, W2: 80 ethanol in water, Elution: water. Mixing and extraction fluidic system consisted of reagent valves, pump and a mixing fixture (FIG. 9 configuration 24b) and 3D capture structures (top-view FIG. 10, configuration 87d and side-view FIG. 11, configuration 87j). Fluidic channel profile was outside these structures a V-cutout. Extraction yield was compared to the same extraction chemistry with manual sample processing. For blood plasma, the average yields were 1.60 and 2.15 (134%) ng/ml relative to plasma for manual and fluidic extraction respectively.

### Example 5

Concentration Measurement of Nucleic Acids Bound on Carboxylated Particles with Fluorometry for Quality Control

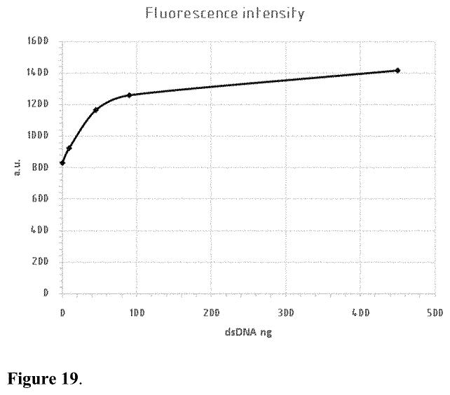

Highly carboxylated M-PVA magnetic particles (nominal diameter 1 μm) were coated with a 119 base pair control DNA fragment at different concentrations (1.5*10⁻⁶-2.4*10⁻⁵ ratio of DNA:COOH, 50-900 ng/mg dsDNA:beads) and fluorescence of the resulting particles was measured in 80% ethanol in water nucleic acid storage solution. The fluorescent dye used was PicoGreen (Invitrogen P11496, final dilution in samples 1:200) and the intensity was recorded with excitation filter at BP470/40 nm and emission filter at BP525/50 nm using either a microplate fluorometer or a fluorescence microscope with a CMOS camera in linear imaging mode. Beads were collected with a permanent magnet to the measurement area formation (see FIG. 14, detail 143 for arrangement and FIG. 18 for a representative fluorescence image). Fluorescence intensity was found to be a suitable method to quantitate the concentration of extracted nucleic acids adsorbed on the carboxylated bead surface in denaturizing solution conditions (FIG. 19).

### Example 6

### Concentration and Size Selection of Small Nucleic Acids Using Sector Gel Electrophoresis

Synthetic sample representing pre-filtered plasma (e.g. filtration using cross-linked dextrans, Sephadex G-10 and G-50) was prepared from 119 bp DNA control fragment, sodium borate running buffer (4.9 mM sodium borate, 16.4 mM boric acid, pH 8.3) and glycerol (5% v/v). Up to 1.5 ml of sample was loaded into 1% agarose gel (gel running device constructed as described in FIG. 8). Collection well containing the elution solution (1500 μl, same composition as running buffer) was isolated from the gel using 0.45 μm Durapore PVDF and 50 kDa PVDF ultrafiltration membranes on the cathode and anode electrode sides respectively. Gel was run in a field of 12 V/cm (210 V) at an average current of 27 mA for 1 hour. Before collection of the concentrate, the anode and cathode potentials were reversed for 3-8 seconds (210 V) or 4-9 minutes at lower voltage (5 V). Recovery yield of DNA fragment was 65-74%.

### Example 7

Separation of Plasma from Preservative-Treated Whole Blood Using Fiber Filtration Including a Cell Wash Step for the Extraction of cfDNA

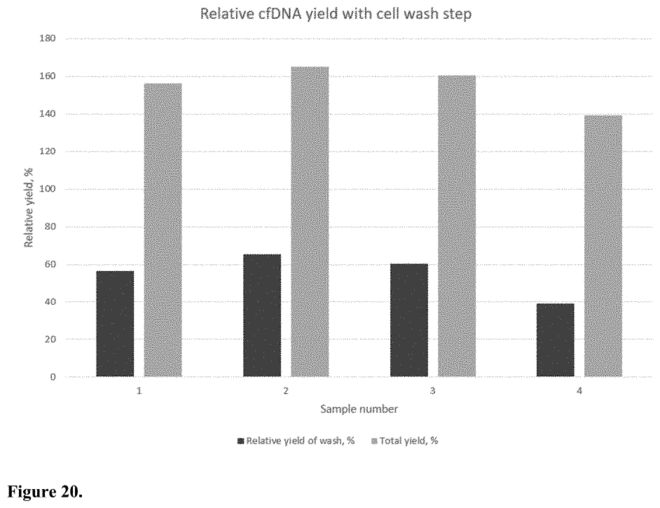

Blood was collected from four individuals into preservative blood collection tubes (Streck cell-free DNA BCT). Plasma separation was performed from the whole blood by passing the sample through a set of polypropylene (average pore size <500 nm) hollow fiber filters with a combined filter surface area of 1800 mm². Fibers were arranged into sets of 20 in parallel with total length of fiber being 1700 mm. Pressure in the plasma compartment outside fibers was −40 mbar relative to the atmosphere. Whole blood sample (8-9 ml) was passed through the filter four times and the separated plasma collected. Blood flow rate was 120 ml/h for gas pressure pumping (positive pressure of 100-400 mbar depending on the filtration cycle number). Additionally, a fifth separation cycle, i.e. a washing step, was performed after mixing the concentrated blood cell fraction with a wash solution (30% glycerol in 1×PBS pH 7.4, volume 2.5 ml). Cell-free DNA was isolated from the prepared plasma filtrate and wash fraction using a commercial extraction kit chemistry (Perkin Elmer NextPrep-Mag) at full sample scale. The size fractioning (i.e. size distribution) of the cfDNA in the wash was confirmed using gel electrophoresis to match that of the sample fraction prepared before the cell wash step. Yield for four randomly selected samples is shown in FIG. 20 where the average combined cfDNA yield, when employing the cell washing step, was found to be 155% relative to the fraction of sample processed without this step, demonstrating improved sample recovery.

### REFERENCES

1. Meddeb, R., Pisareva, E., Thierry A. R. Guidelines for the Preanalytical Conditions for Analyzing Circulating Cell-Free DNA. Clinical Chemistry (2019) 65 (5) 623-633;
2. Ashoor, G. Syngelaki, A. Poon, L. C. Y. Rezende, J. C., Nicolaides K. H. Fetal fraction in maternal plasma cell-free DNA at 11-13 weeks' gestation: relation to maternal and fetal characteristics. Ultrasound Obstet Gynecol. (2013) 41(1): 26-32.
3. Diaz L A Jr, Bardelli A. Liquid biopsies: genotyping circulating tumor DNA. J Clin Oncol. (2014) 32:579-86. doi: 10.1200/JCO.2012.45.2011
4. Diehl F, Schmidt K, Choti M A, Romans K, Goodman S, Li M, et al. Circulating mutant DNA to assess tumor dynamics. Nat Med. (2008) 14:985-90. doi: 10.1038/nm.1789
5. Kidess E, Jeffrey S S. Circulating tumor cells versus tumor-derived cell-free DNA: rivals or partners in cancer care in the era of single-cell analysis? Genom Med. (2013) 5:70. doi: 10.1186/gm474
6. Mouliere, Florent & Piskorz, Anna & Chandrananda, Dineika & Moore, Elizabeth & Morris, James & Smith, Christopher & Goranova, Teodora & Heider, Katrin & Mair, Richard & Supernat, Anna & Gounaris, Ioannis & Ros, Susana & Wan, Jonathan & Jimenez-Linan, Mercedes & Gale, Davina & Brindle, Kevin & Parkinson, Christine & Brenton, James & Rosenfeld, Nitzan. (2017). Selecting Short DNA Fragments In Plasma Improves Detection Of Circulating Tumour DNA. 10.1101/134437.
7. Abbosh C, Birkbak N J, Wilson G A, et al. Phylogenetic ctDNA analysis depicts early-stage lung cancer evolution. Nature (2017) 545:446-51.
8. Sina, A. A. I., Carrascosa, L. G., Liang, Z. et al. Epigenetically reprogrammed methylation landscape drives the DNA self-assembly and serves as a universal cancer biomarker. Nat Commun 9, 4915 (2018) doi:10.1038/s41467-018-07214-w
9. Lo Y M, Zhang J, Leung T N, Lau T K, Chang A M, Hjelm N M. Rapid clearance of fetal DNA from maternal plasma. Am J Hum Genet. (1999) 64:218-24. doi: 10.1086/30§ 05
10. Saarenheimo, J., Eigeliene, N., Andersen, H., Tiirola, M., Jekunen, A. The value of liquid biopsies for guiding therapy decisions in non-small cell lung cancer. Frontiers in Oncology (2019) 9:129.
11. Molina-Vila M A, de-Las-Casas C M, Bertran-Alamillo J, Jordana-Ariza N, González-Cao M, Rosell R. cfDNA analysis from blood in melanoma. Ann Transl Med. (2015) 3:309.
12. 10. R. M. Trigg, L. J. Martinson, S. Parpart-Li, J. A. Shaw. Factors that influence quality and yield of circulating-free DNA: A systematic review of the methodology literature. Heliyon 4 (2018) e00699. doi: 10.1016/j.heliyon.2018. e00699
13. Sisson, B. A., Uvalic, J., Kelly, K., Selvam, P., Hesse, A. N., Ananda, G., . . . Reddi, H. V. Technical and Regulatory Considerations for Taking Liquid Biopsy to the Clinic: Validation of the JAX PlasmaMonitor™ Assay. Biomarker Insights (2019) https://doi.org/10.1177/1177271919826545
14. Merker J D, Oxnard G R, Compton C, et al. Circulating Tumor DNA Analysis in Patients With Cancer: American Society of Clinical Oncology and College of American Pathologists Joint Review. J Clin Oncol (2018) 36(16):1631-41.
15. Wang, B. G., H. Y. Huang, Y. C. Chen, R. E. Bristow, K. Kassauei, C. C. Cheng, R. Roden, L. J. Sokoll, D. W. Chan, and I. Shih. Increased plasma DNA integrity in cancer patients. Cancer Res. (2003) 63:3966-3968.
16. Mouliere, F., B. Robert, E. Arnau Peyrotte, M. Del Rio, M. Ychou, F. Molina, C. Gongora, and A. R. Thierry High fragmentation characterizes tumour-derived circulating DNA. PLoS One. (2011) 6:e23418.
17. Torga G, Pienta K J. Patient-paired sample congruence between 2 commercial liquid biopsy tests. JAMA Oncol. (2018) 4:868-870.
18. Hu F., Li J., Peng N., Li Z., Zhang Z., Zhao S., Duan M., Tian H., Lia L. and Zhanga P. Rapid isolation of cfDNA from large-volume whole blood on a centrifugal microfluidic chip based on immiscible phase filtration. Analyst, (2019) 144, 4162-4174 doi:10.1039/C9AN00493A

### PATENT DOCUMENTS

EP2315849
U.S. Pat. No. 4,234,431
U.S. Pat. No. 5,863,801A
US20040050699
WO2016061416
U.S. Pat. No. 6,802,820
U.S. Pat. No. 6,802,971
WO2019185874
WO2019136086

1. A method for extraction of nucleic acid fragments from a blood sample comprising the steps of:
a) providing a blood sample taken from an individual;
b) stabilizing blood cells in the sample with a fixative reagent;
c) filtering the blood sample in order to separate plasma from said blood sample by using a hollow fiber filter;
d) contacting the plasma sample obtained in step c) with a binder material specific to nucleic acids, or alternatively subjecting the plasma sample to electrophoresis in a separating medium, in order to purify nucleic acid fragments present in said plasma sample, wherein said separating medium is prepared so that nucleic acid fragments are capable of migrating in the medium and can be separated in said medium by size;
e) collecting those nucleic acid fragments bound to said binder material or which have migrated in said separating medium; and
f) mixing the nucleic acid fragments collected in step e) with a preservative or a stabilizing agent;
wherein the steps b) to f) are performed on an automated system on a cartridge comprising a first compartment for filtering plasma from a blood sample and a second compartment for contacting the plasma sample obtained in step c) with a binder material specific to nucleic acids, or alternatively said second compartment comprises means for performing an electrophoresis.

2. The method according to claim 1, wherein in step d) the obtained mix of the nucleic acid fragments and said preservative or a stabilizing agent is stored into a removable container.

3. The method according to claim 2, wherein said mix comprises said nucleic acid fragments bound to said binder material.

4. The method according to claim 1, wherein the length of said nucleic acid fragments is less than 400 bp.

5. The method according to claim 1, wherein the blood sample obtained in step a) is mixed with an anticoagulant before subjecting the sample to the filtering step c).

6. The method according to claim 1, wherein said hollow fiber filter is a hollow fiber membrane.

7. The method according to claim 1, wherein in step d) the plasma sample is contacted with a binder material specific to nucleic acids.

8. The method according to claim 7, wherein said binder material comprises magnetic beads.

9. The method according to claim 1, further comprising contacting the nucleic acid fragments bound to said binder material with a nucleic acid binding dye and measuring the amount of collected nucleic acid fragments.

10-12. (canceled)

13. A cartridge comprising a first compartment for filtering plasma from a blood sample and a second compartment for contacting a plasma sample with a binder material specific to nucleic acids, wherein the first compartment comprises a hollow fiber filter and the second compartment comprises a chamber for nucleic acid purification, and said cartridge comprises a binder material specific to nucleic acids encapsulated in said cartridge, wherein said binder material comprises magnetic beads.

14. The cartridge according to claim 13, wherein said first compartment comprises means for receiving a blood sample.

15. The cartridge according to claim 14, wherein said first compartment comprises means for mixing said blood sample with a cell fixation reagent encapsulated in said first compartment, wherein said first compartment comprises means for washing the cells filtered from said blood sample, and wherein a wash buffer is encapsulated in said cartridge.

16. The cartridge according to claim 13, wherein said first compartment comprises means for mixing a plasma sample separated from said blood sample in a hollow fiber membrane with an enzymatic or chemical reagent encapsulated in said first compartment.

17. The cartridge according to claim 13, wherein said first compartment comprises means for transferring the separated plasma mixed with said enzymatic or chemical reagent from the first compartment to the second compartment.

18. The cartridge according to claim 13, wherein the cartridge comprises a preservative or a stabilizing agent encapsulated in said cartridge, means for mixing said preservative or a stabilizing agent with nucleic acid fragments bound to said binder material, and a removable container for storing the mix of the nucleic acid fragments and a preservative or a stabilizing agent.

19. The cartridge according to claim 13, wherein the cartridge comprises a nucleic acid binding dye encapsulated in said cartridge.

20. The cartridge according to claim 19, wherein the second compartment comprises a transparent window arranged to direct a beam of light through the sample or mix to measure the amount of isolated nucleic acid fragments bound to said dye by optical means.

21. An automated system for extraction of nucleic acid fragments from a blood sample comprising a device with a docking site adapted to receive the cartridge according to claim 13, said device comprising means adapted to operate the blood plasma filtering process in said cartridge and means adapted to operate nucleic acid purification in said cartridge with a binder material specific to nucleic acids.

22-42. (canceled)
